(19) United States
(12) Patent Application Publication
(10) Pub. No.: US 2025/0064758 A1
(43) Pub. Date: Feb. 27, 2025

(54) ANTIVIRALS TARGETING PHOSPHATIDIC ACID PHOSPHATASE (PAP)

(71) Applicants: The University of Hong Kong, Hong Kong (CN); Centre for Virology, Vaccinology and Therapeutics Limited, Hong Kong (CN)

(72) Inventors: Bingpeng Yan, Hong Kong (CN); Jasper Fuk-Woo Chan, Hong Kong (CN); Yi Tsun Richard Kao, Hong Kong (CN); Shuofeng Yuan, Hong Kong (CN); Cynthia Cheuk Ying Shum, Hong Kong (CN)

(21) Appl. No.: 18/813,853

(22) Filed: Aug. 23, 2024

Related U.S. Application Data
(60) Provisional application No. 63/578,582, filed on Aug. 24, 2023.

(51) Int. Cl. A61K 31/138; A61K 45/06; A61P 31/14

(52) U.S. Cl. CPC A61K 31/138; A61K 45/06; A61P 31/14

(57) ABSTRACT

Host-targeting antiviral agents against conserved phosphatidic acid phosphatase (PAP) pathways that act as broad-spectrum treatment strategies for different coronaviruses, such as variants of SARS-COV-2, have been developed. Compositions of anti-coronavirus agent(s) that acts via the host PAP pathway and methods of use thereof for treating and preventing diseases and disorders associated with coronaviruses are provided. An exemplary anti-coronavirus agent that acts via the PAP pathway is propranolol. Compositions of propranolol and methods of use thereof for treating and preventing diseases and disorders associated with coronaviruses are provided. In some forms, the compositions and methods treat or prevent one or more symptoms associated with infection by SARS-COV-2 wild type, Delta variants, Omicron variants and MERS-COV in a subject in need thereof.

### CROSS REFERENCE TO RELATED APPLICATIONS

This application is claims priority to and the benefit of U.S. Provisional Application No. 63/578,582, filed Aug. 24, 2023, the content of which is hereby incorporated by reference herein in its entirety.

### FIELD OF THE INVENTION

The invention is generally directed to compositions and methods for targeting host factors for preventing or treating coronavirus infections, and particularly to targeting the phosphatidic acid phosphatase (PAP) pathway for treating coronaviruses including SARS-CoV-2 and variants such as Omicron and Delta.

### BACKGROUND OF THE INVENTION

Coronaviruses have caused three outbreaks (2003-SARS-COV, 2012-MERS-COV, and 2019-SARS-COV-2) in the past twenty years. The SARS-COV-2 pandemic has lasted for almost 2 years at the time of writing (J. F. Chan, et al., Lancet, 395 (2020) 514-523). However, there are few widely available drugs, which could effectively protect humans from SARS-COV-2 infection. Patients with severe COVID-19 pneumonia have diffuse alveolar damage with syncytia formation in their lung tissue, which is attributed to viral spike-ACE2 mediated cell fusion (R. Bussani, et al., EBioMedicine, 61 (2020) 103104; Z. Xu, et al., Lancet Respir Med, 8 (2020) 420-422). The syncytia caused by viral spike-ACE2 mediated fusion might be related to the excessive inflammatory responses in severe COVID-19 patients and anti-syncytia drugs might yield better clinical outcomes (H. Ma, et al., Cell Discov, 7 (2021) 73; J. Buchrieser, et al., Embo j, 39 (2020) e 106267; and L. Lin, et al., Cell Death Differ, 28 (2021) 2019-2021).

Many studies have tried to identify viral entry inhibitors as the key antiviral strategy (R. Heida, et al., Drug Discov Today, 26 (2021) 122-137). SARS-COV-2 is known to enter cells by binding to heparan sulfate (HS) and the receptor ACE2 which allows cell entry through TMPRSS2-mediated cell membrane fusion pathway or endocytosis pathway (T. M. Clausen, et al., Cell, 183 (2020) 1043-1057.e1015; and M. Hoffmann, et al., Cell, 181 (2020) 271-280 e278). Though many studies have shown that some antivirals could block SARS-COV-2 entry or viral RNA synthesis (D. Asarnow, et al., Cell, 184 (2021) 3192-3204.e3116; V. Gil Martínez, et al., Pharmaceuticals (Basel), 2021 Jul. 28; 14 (8): 736; Y. W. Zhou, et al., Signal Transduct Target Ther, 6 (2021) 317), reports on how SARS-COV-2 is released from infected cells to enter other uninfected cells are limited. Moreover, no antiviral has yet been clearly demonstrated to inhibit SARS-CoV-2 release.

As SARS-COV-2 continues to adapt in humans, variants with altered pathogenicity and/or transmissibility have appeared. The most recently emerged Omicron (BA.5) variant is characterized by an unusually high number of amino acid mutations at its spike protein which renders it to be less susceptible to the neutralizing antibody response elicited by previous COVID-19 vaccines (Iketani, et al. Antibody evasion properties of SARS-COV-2 Omicron sublineages. Nature. 2022.). Variant viruses exploit the host lipid metabolism machinery to achieve efficient replication. Hence, virus-targeting antivirals such as monoclonal antibodies and enzyme inhibitors may become ineffective when resistant virus strains emerge (Zumla, et al., Coronaviruses—drug discovery and therapeutic options. Nature reviews Drug discovery. 2016; 15 (5): 327-47).

There is a need for developing effective prophylactic and/or therapeutic therapy that acts against respiratory pathogens such as SARS-COV-2 by targeting pathways or proteins that are less likely to become adapted for drug resistance.

Therefore, it is an object of the invention to provide broad-spectrum and long-lasting prophylactic and/or therapeutic agents for treating and/or preventing new and emerging SARS-COV-2 infections.

It is another object of the invention to provide compositions and methods for treating and/or preventing one or more of the pathological processes associated with Omicron and/or Delta SARS-COV-2 infections in a host.

### SUMMARY OF THE INVENTION

It has been discovered that phosphatidic acid phosphatase (PAP) is an essential host factor for efficient SARS-COV-2 replication. Compositions and methods targeting the host PAP enzyme and associated pathways inhibit the replication of multiple coronaviruses including SARS-COV-2 wild type, variants, and MERS-COV at the cell level. Therefore, compositions and methods for targeting the host PAP enzyme and associated pathways are provided for treating and preventing one or more symptoms of coronavirus infection in a subject in need thereof. An exemplary anti-coronavirus agent(s) that acts via the host PAP pathway is propranolol. Therefore, compositions of propranolol, or a derivative or variant of propranolol in an amount effective for treating or preventing one or more symptoms of coronavirus infection in a subject in need thereof are provided.

Also provided are methods of treating, retarding development of, or preventing development of one or more symptoms of coronavirus infections, and/or diseases and disorders associated with coronavirus infection by administering to a subject in need thereof an effective amount of the anti-coronavirus agent(s), such as propranolol. The methods are particularly suited for use in a subject is having a coronavirus infection or at risk of contracting a coronavirus associated disease. In some forms, the coronavirus is SARS-COV-2 virus or a variant thereof, for example, SARS-COV-2 B.1.1.7 (Alpha variant), SARS-COV-2 B.1.351 (Beta variant), SARS-COV-2 P.1 (Gamma variant), SARS-COV-2 B.1.617, SARS-COV-2 B.1.617.1 (Kappa variant), SARS-COV-2 B.1.621 (Mu variant), SARS-COV-2 B.1.617.2 (Delta variant), SARS-COV-2 B.1.617.3, or SARS-CoV-2 B.1.1.529 (Omicron variant).

The methods generally administer the composition to a subject in need thereof in an amount effective to reduce or prevent the replication of the coronavirus in the subject as compared to a control.

In some forms, the composition is administered to a human subject at a dose of between 0.1 mg/kg body weight of the subject and 10 mg/kg body weight of the subject, inclusive; or at a dose of between 10.0 mg and 320 mg per day, inclusive; optionally at a dose of 120 mg per day. Preferably, the methods administer the composition in an amount effective to reduce one or more symptoms of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

In some forms, the methods of reducing coronavirus replication in a cell involve contacting the cell with an anti-coronavirus agent, where the cell includes a replicative coronavirus genome, where the anti-coronavirus agent inhibits one or more gene expression products associated with the phosphatidate phosphatase (PAP) pathway, and where the amount of the anti-coronavirus agent contacted with the cell is effective to reduce the replication of the coronavirus in the cell relative to an untreated control cell.

In some forms, the methods of treating or preventing one or more symptoms of a coronavirus infection in a subject involve administering to the subject an anti-coronavirus agent, where the anti-coronavirus agent inhibits one or more gene expression products associated with the phosphatidate phosphatase (PAP) pathway, and where the amount of the anti-coronavirus agent administered to the subject is effective to reduce the replication of the coronavirus in the cell relative to an untreated control cell.

In some forms, the anti-coronavirus agent includes propranolol, or a derivative or variant of propranolol.

### BRIEF DESCRIPTION OF THE DRAWINGS

FIGS. 1A-1D are bar graphs showing antiviral activity of Propranolol as a function of the fitness of SARS-COV-2 (wild type) virus in multiple cell lines. FIGS. 1A-1C are bar graphs showing dose-response analysis of propranolol in Calu-3 (FIG. 1A), Caco-2 (FIG. 1B), and VeroE6-TMPRSS2 cells (FIG. 1C), respectively, also depicting the IC50 value of the propranolol in each cell line. Calu-3 was infected by 0.5 MOI SARS-COV-2. Viral load in Calu-3 cell supernatant was collected at the 48 hpi and determined by RT-qPCR assays. Caco-2 and VeroE6-TMPRSS2 cells were infected by 0.1 MOI SARS-COV-2. Viral load in the Caco-2 lysate and VeroE6cell supernatant were collected at the 24 hpi and determined by RT-qPCR assays. FIG. 1D is a bar graph showing Propranolol inhibition of SARS-COV-2 replication in human embryonic stem cells-derived cardiomyocytes (hES-CMs) that were infected by 0.1 MOI SARS-COV-2, showing virus genome copies (% of control), for each of 50 uM Propranolol (1), 10 uM Remdesivir (2), and DMSO (3) in each of intracellular (Cell lysate), and extracellular (cell supernatant) samples, respectively. Viral load in the cell lysate and cell supernatant were collected at the 24 hpi and determined by RT-qPCR assays, respectively. Data represent mean±SD. One-way ANOVA when compared with the DMSO control group (n=3). For all statistical analysis above, *p<0.05, **p<0.01 and ***p<0.001.

FIGS. 2A-2F are graphs showing the effects of Propranolol on SARS-COV-2 virus (multiple variants) in VeroE6-TMPRSS2 cells. FIGS. 2A-2C are curved graphs of the effect of Propranolol concentration (0-100 uM) on viral copies (% control) in each of Delta variant B.1.617.2 virus (FIG. 2A); Omicron variant BA.4 virus (FIG. 2B); and Omicron variant BA.5 virus (FIG. 2C), respectively. Data are used to generate the half maximal inhibitory concentration (IC50) curves. FIGS. 2D-2F are histograms showing the effect of Propranolol concentration (uM) on viral gene copy number in each of Delta variant B.1.617.2 virus (FIG. 2D), Omicron variant BA.4 virus (FIG. 2E), and Omicron variant BA.5 virus (FIG. 2F) infected cells, respectively. The VeroE6-TMPRSS2 cells were infected by 0.1 MOI SARS-COV-2 variants and viral gene copies in the cell supernatant were determined at 24 hpi by RT-qPCR assays, respectively. Data represent mean±SD. One-way ANOVA, multiple comparisons test with p-values corrected according to Dunnett's test. IC50 curves obtained by nonlinear regression fit calculated on Prism 7. For all statistical analysis above, *p<0.05, **p<0.01 and ***p<0.001.

FIGS. 3A-3F are graphs showing the effects of Propranolol on other coronaviruses (e.g., MERS-COV, SARS-COV and HCoV-229E) in various cell types. FIGS. 3A-3B are graphs showing the half maximal inhibitory concentration (IC50) curves, demonstrating Propranolol's antiviral potency in MERS-COV-infected cells, depicting viral gene copies (FIG. 3A) and live virus (FIG. 3B), over Propranolol concentration (0-100 μM), respectively. FIGS. 3C-3D are IC50 graphs showing Propranolol inhibited SARS-COV replication in VeroE6-TMPRSS2 cells. Viral gene copies (FIG. 3C) in the cell supernatant and live virus amount (PFU/ml (% control); FIG. 3D) were determined at 24 hpi by RT-qPCR assays and plaque assay, respectively. The VeroE6-TMPRSS2 cells were infected with either MERS-COV or SARS-COV at 0.1 MOI. FIGS. 3E-3F are IC50 graphs showing the effects of Propranolol on the replication of HCoV-229E in Huh7 cells, showing viral copies as a % of control as determined from each of intracellular HCoV-229E in cell lysate (FIG. 3E); and extracellular HCoV-229E in supernatant samples (FIG. 3F), respectively, as determined by RT-qPCR assays. Data represent mean±SD. One-way ANOVA, multiple comparisons test with p-values corrected according to Dunnett's test. IC50 curves obtained by nonlinear regression fit calculated on Prism 7.

FIGS. 4A-4C are bar graphs showing the antiviral effect evaluation of other beta blockers and agonists. The antiviral effects were evaluated by other beta blockers such as Nadolol (FIG. 4A), Atenolol (FIG. 4B) and beta receptor agonist such as Isoproterenol (FIG. 4C) in VeroE6-TMPRSS2 cells that were infected by 0.1 MOI Omicron variant BA.5. Viral gene copies in the cell supernatant were determined at 24 hpi by RT-qPCR assays. One-way ANOVA, multiple comparisons test with p-values corrected according to Dunnett's test.

FIGS. 5A-5B are images of Western Blot showing results of a co-immunoprecipitation (Co-IP) assay for investigating the potential interaction of host protein and viral protein. Input (FIG. 5A) and Co-IP blot results (FIG. 5B) show 5 lanes, each having the presence (+) or absence (−) of SARS-COV2 nucleocapsid protein (NP), lipin1 protein, and lipin3 protein, respectively. The relative positions of 46 kDa and 130 kDa markers are also indicated. The host-viral protein-protein interaction was evaluated with co-immunoprecipitation assays using SARS-COV2 NP as a bait protein. The current results revealed that either the lipin1 or lipin3 bands were observed as a pull-down product of NP, indicating an interaction between lipin1/lipin3 and SARS-COV-2 NP.

FIG. 6A is a schematic of the experimental design of the propranolol regimen against WT SARS-COV-2. FIGS. 6B-6C are graphs showing the in vivo efficacy evaluation of propranolol on SARS-COV-2 infection by using established golden Syrian hamster model. Notably, propranolol decreased WT SARS-COV-2 genome copies (p<0.05, FIG. 6B) and infectious virus titer (p<0.01, FIG. 6C) in comparison with the mock-treated samples in lung tissues.

### DETAILED DESCRIPTION OF THE INVENTION

### I. Definitions

The terms “individual”, “host”, “subject”, and “patient” are used interchangeably, and refer to a mammal, including, but not limited to, murines, simians, humans, mammalian farm animals, mammalian sport animals, and mammalian pets.

The term “effective amount” or “therapeutically effective amount” refers to the amount which is able to treat one or more symptoms of a disease or disorder, reverse the progression of one or more symptoms of a disease or disorder, halt the progression of one or more symptoms of a disease or disorder, or prevent the occurrence of one or more symptoms of a disease or disorder in a subject to whom the formulation is administered, for example, as compared to a matched subject not receiving the compound. The actual effective amounts of compound can vary according to the specific compound or combination thereof being utilized, the particular composition formulated, the mode of administration, and the age, weight, condition of the individual, and severity of the symptoms or condition being treated.

The term “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier is “acceptable” in the sense of being compatible with the other ingredients of a subject composition and not injurious to the patient.

The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluene-sulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; etc.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, compositions including therapeutic agents may inhibit or reduce one or more markers of a disease or disorder in a subject by about 10%, 20%, 30%, 40%, 50%, 75%, 85%, 90%, 95%, or 99% from the activity and/or quantity of the same marker in subjects that did not receive or were not treated with the compositions. In some forms, the inhibition and reduction are compared according to the level of mRNAs, proteins, cells, tissues and organs.

The terms “treating” or “retarding development of” in the context of a disease or disorder mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with a coronavirus infection are mitigated or eliminated, including, but are not limited to, reducing and/or inhibiting the syncytial formation and lung damage, increasing the quality of life of those suffering from the disease, decreasing the dose of other medications required to treat the disease, delaying the progression of the disease, and/or prolonging survival of individuals.

The term “biodegradable” generally refers to a material that will degrade or erode under physiologic conditions to smaller units or chemical species that are capable of being metabolized, eliminated, or excreted by the subject. The degradation time is a function of composition and morphology.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide.

The terms “Coronavirus disease 2019”, “COVID-19”, or “COVID” refer to the disease caused by the human pandemic SARS-COV-2 virus.

The term “combination therapy” refers to treatment of a disease or symptom thereof, or a method for achieving a desired physiological change, including administering to an animal, such as a mammal, especially a human being, an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

The term “dosage regime” refers to drug administration regarding formulation, route of administration, drug dose, dosing interval and treatment duration.

Recitation of ranges of values herein are merely intended to serve as a shorthand method of referring individually to each separate value falling within the range, unless otherwise indicated herein, and each separate value is incorporated into the specification as if it were individually recited herein.

### II. Compositions

It has been established that inhibition of the phosphatidic acid phosphatase (PAP) pathway, for example, via direct or indirect inhibition of one or more phosphatidic acid phosphatase enzymes broadly inhibited replication of SARS-COV-2 variants in vitro and in vivo.

Mechanistic studies in the Examples show that active agents that inhibit the phosphatidic acid phosphatase-1 enzyme (PAP-1) inhibits the replication of multiple coronaviruses including SARS-COV-2 wild type, variants, and MERS-COV at the cell level. Exemplary agents that are active against PAP include propranolol, as well as derivatives and variants thereof.

### A. PAP-Pathway Antagonists

The lipin protein family of phosphatidate phosphatases has an established role in triacylglycerol synthesis and storage. Physiological roles for lipin-1 and lipin-2 have been identified, but the role of lipin-3 has remained mysterious.

The enzyme phosphatidate phosphatase (PAP, EC 3.1.3.4) is a key regulatory enzyme in lipid metabolism, catalyzing the conversion of phosphatidate to diacylglycerol. The reverse conversion is catalyzed by the enzyme diacylglycerol kinase, which replaces the hydroxyl group on diacyl-glycerol with a phosphate from ATP, generating ADP in the process. When phosphatidate phosphatase is inactive, diacylglycerol kinase catalyzes the reverse conversion, allowing phosphatidate to accumulate as it brings down diacylglycerol levels. Phosphatidate can then be converted into an activated form, CDP-diacylglycerol by liberation of a pyrophosphate from a CTP molecule, or into cardiolipin, a principal precursor in phospholipid synthesis.

There are several different genes that code for phosphatidate phosphatases. They fall into one of two types (type I and type II), depending on their cellular localization and substrate specificity.
Type I phosphatidate phosphatases (PAP1) are soluble enzymes encoded by the Lipin genes and are substrate-specific only to phosphatidate. The lipin enzymes (Lipin1, Lipin2, and Lipin3) are speculated to be involved in the de novo synthesis of glycerolipids in mammals, with distinct physiological functions and tissue expression motifs.
Type-II phosphatidate phosphatases enzymes (PAP2; Diacylglycerol diphosphate phosphatase) are plasma membrane-bound, Mg2+-independent enzymes involved in lipid mediated signal transduction and metabolism. PAP2 binds phosphatidic acid via the catalytic domain, and is involved in a diverse range of processes such as vesicular secretion, endocytosis, recruitment of enzymes, etc. The action of PAP2 is, therefore, closely linked to the maintenance of PA homeostasis.

It has been established that antagonists of the PAP enzymes and/or PAP pathway inhibit and/or prevent coronavirus replication. Therefore, antiviral compositions of PAP-pathway antagonists that have reduce or prevent coronavirus infection and/or replication are provided.

It has been proposed that Lipin-1 and lipin-3 cooperate in vivo to determine adipose tissue PAP activity and adiposity. Therefore, in some forms, compositions of antiviral PAP-Pathway antagonists include one or more inhibitors of lipin1 and/or lipin3.

It has been proposed that the catalytic acid phosphatase domain (APD) of PAP2 enzymes are effectively inhibited by interaction with small-molecule inhibitors. Therefore, in some forms, compositions of antiviral PAP-Pathway antagonists include one or more inhibitors of the PAP2 enzyme catalytic domain. Exemplary agents that act via binding to, and thereby blocking the molecular activity of the mammalian PAP2 APD is or includes propranolol, as well as derivatives and variants thereof.

### 1. Propranolol

In some forms, the antiviral PAP pathway inhibitor is or includes propranolol. Phosphatidic acid (PA) and propranolol both bind to the same active site on the predicted catalytic domain of PAP2, and it is contemplated that binding of propranolol to PAP2 inhibits PAP1 and/or PAP2 enzyme function and thereby precludes host PAP-pathway activity.

As demonstrated in the Examples, Propranolol reduced SARS-COV-2 wild type gene copies in the culture supernatant at non-toxic concentrations in multiple cell lines, including human cells. Propranolol treatment diminished both extracellular and intracellular SARS-COV-2 viral load remarkably in similar magnitude as that of remdesivir.

Propranolol [1-(isopropyl amino)-3-(1-naphthyloxy)-2-propanol], also known as 2-Propanol, 1-[(1-methylethyl)amino]-3-(1-naphthalenyloxy) is a beta-adrenergic blocking agent and a potent inhibitor of the PAP activity of lipins, widely used in clinic for a variety of diseases, and it has minimal toxicity, with little to no effect on normal tissues at the clinically effective dose.

The chemical structure of propranolol is set forth in Formula I, below:

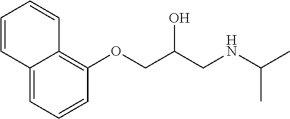 [SMILES: CC(C)NCC(O)COc1cccc2ccccc12]

Formula I: Propranolol (C16H21NO2; Molecular Mass 259.34; CAS:525-66-6)

By inhibiting the PAP activity of lipins, propranolol blocks the fusion between lysosome and autophagosome, which leads to the massive accumulation of immature autophagosomes and to an overall blockage of autophagy. This blockage of autophagy leads to the aggravation of ER stress, a mechanism widely exploited and very efficient in therapeutic regimens.

Propranolol is widely distributed into body tissues including lungs, liver, kidneys, and heart. Propranolol readily crosses the blood-brain barrier and the placenta and is distributed into milk. The apparent volume of distribution of propranolol at steady state varies widely in proportion to the fraction of unbound drug in whole blood. Propranolol is more than 90% bound to plasma proteins over a wide range of blood concentrations. Both free and protein-bound propranolol are metabolized. Increased plasma protein binding of the drug increases its metabolism and decreases its volume of distribution, resulting in a shorter terminal half-life. Oral propranolol is useful therapeutically because it is lipid soluble and readily absorbed from the small intestine; however, because of extensive first-pass hepatic metabolism, larger oral doses are required to achieve clinical effects equivalent to those of intravenous doses.

Propranolol and methods of making propranolol is described in U.S. Pat. Nos. 3,337,628, 3,520,919, 6,121,328 and 10,828,265, the contents of which are all incorporated by reference herein in their entirety. In some forms, the PAP-pathway antagonist is a small molecule variant or derivative of propranolol. A thorough description of the optical isomers of propranolol is found in an article by Howe and Shanks, Nature 210,1336 (1966). A study of the metabolism of the compound is found in an article by Bon, Nature 213, 721 (1967). P. A. Routledge and D. G. Shand review the pharmacokinetics of propranolol, Appl. Pharmacokinet. (1980), 464-485.

The elimination half-life of propranolol is approximately 8 hours. The plasma half-life of propranolol is 3 to 6 hours. The clearance of propranolol is 2.7±0.03 L/h/kg in infants <90 days and 3.3±0.35 L/h/kg in infants >90 days. Propranolol clearance increases linearly with hepatic blood flow.

The D and L isomers have distinct activities, such that the “D” isoform has beta-blocking efficacy, whereas the “L” isoform does not have this activity. Therefore, in some forms, the propranolol is or includes the L isoform of propranolol and has little or no beta-blocking activity in a subject when administered to the subject in vivo. In other forms, the propranolol is or includes the D isoform of propranolol and has beta blocking activity in a subject when administered to the subject in vivo.

Propranolol and formulations thereof are commercially available, sold under many trademarks, including INDERAL®. In some forms, formulations of Propranolol contain a mixture of 50% D and 50% L propranolol. In other forms, the propranolol is a pure D isoform. In other forms, the propranolol is a pure L isoform. In other forms, the propranolol is a mixture including from 99.9% to 0.01% of the total amount, by weight, being L or D isoform.

i. Propranolol Derivatives

In some forms, the active agent is a derivative or analog of propranolol. Typically, the derivatives or analogs of propranolol effectively bind to and inhibit the phosphatidic-acid binding site of host PAP2 enzymes, and or reduce or inhibit the host PAP pathway.

Derivatives of propranolol are known in the art, for example, as described in Tran, et al., Journal of Chemistry, 2020, (Article ID 9597426); Groszek, et al., Molecules 2010, 15(6), 3887-3904; and Mohammed, et al., Pharmacie Globale (IJCP) 2013, 07 (03), the contents of which are incorporated by reference herein in their entirety.

Exemplary derivatives of propranolol are formed by reactions at the C-13 and N-15 position. Exemplary reactions that give rise to derivatives of propranolol include esterification reactions, for example, between propranolol and 2-bromobenzoyl chloride, 2-chlorobenzoyl chloride, or 2-fluorobenzoyl chloride, respectively.

The chemical structure of exemplary propranolol derivatives include the following structures set forth in Formulas II-IV, below:

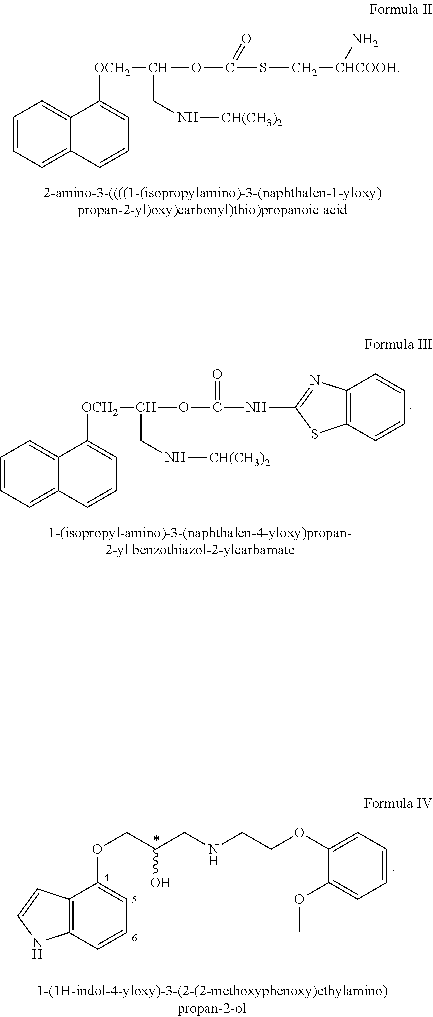

In some forms, the compositions of anti-coronavirus agent(s) that acts via the host PAP pathway, such as propranolol, include one or more additional therapeutic, prophylactic or diagnostic agents.

One or more additional therapeutic, diagnostic, and/or prophylactic agents may be used to treat or retard development of, or prevent development of inflammation in the lungs, and/or systemic inflammation resulting from COVID-19 induced pneumonia.

In addition to the anti-coronavirus agent(s) that acts via the host PAP pathway, such as propranolol, the composition can contain one or more additional therapeutic, diagnostic, and/or prophylactic agents that has the same or different target, or the same or different activity against the same or different pathogen. In some forms, the composition contain one or more additional compounds to relieve symptoms such as inflammation, or shortness of breath. Representative therapeutic (including prodrugs), prophylactic, or diagnostic agents are peptides, proteins, carbohydrates, nucleotides or oligonucleotides, small molecules, or combinations thereof. The active agents can be a small molecule active agent or a biomolecule, such as an enzyme or protein, polypeptide, or nucleic acid. Suitable small molecule active agents include organic and organometallic compounds. In some instances, the small molecule active agent has a molecular weight of less than about 2000 g/mol, preferably less than about 1,500 g/mol, most preferably less than about 1,200 g/mol. The small molecule active agent can be a hydrophilic, hydrophobic, or amphiphilic compound. In some cases, one or more additional active agents may be dissolved or suspended in the pharmaceutically acceptable carrier.

In the case of pharmaceutical compositions for the treatment of lung diseases, the formulation may contain one or more therapeutic agents to treat, prevent or diagnose a disease or disorder of the lung. Non-limiting examples of therapeutic agents include bronchodilators, corticosteroids, methylxanthines, phosphodiesterase-4 inhibitors, anti-angiogenesis agents, antibiotics, antioxidants, anti-viral agents, anti-fungal agents, anti-inflammatory agents, immunosuppressant agents, anti-allergic agents, and combinations thereof.

The amount of a second therapeutic generally depends on the severity of the coronavirus-related diseases and/or disorders to be treated. Specific dosages can be readily determined by those of skill in the art. See Ansel, Howard C. et al. Pharmaceutical Dosage Forms and Drug Delivery Systems (6^{th} ed.) Williams and Wilkins, Malvern, PA (1995).

In other forms, one or more agents include bronchodilators, corticosteroids, methylxanthines, phosphodiesterase-4 inhibitors, anti-angiogenesis agents, antibiotics, antioxidants, anti-viral agents, anti-fungal agents, anti-inflammatory agents, immunosuppressant agents, and/or anti-allergic agents, are administered prior to, in conjunction with, subsequent to, or alternation with treatment with the disclosed formulation of anti-coronavirus agent(s) that acts via the host PAP pathway, such as propranolol.

The additive drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

In some forms, the additional agent is a diagnostic agent imaging or otherwise assessing the site of application. Exemplary diagnostic agents include paramagnetic molecules, fluorescent compounds, magnetic molecules, and radionuclides, x-ray imaging agents, and contrast media. These may also be ligands or antibodies which are labelled with the foregoing or bind to labelled ligands or antibodies which are detectable by methods known to those skilled in the art.

In certain forms, the pharmaceutical composition contains one or more local anesthetics. Representative local anesthetics include tetracaine, lidocaine, amethocaine, proparacaine, lignocaine, and bupivacaine. In some forms, one or more additional agents, such as a hyaluronidase enzyme, is also added to the formulation to accelerate and improves dispersal of the local anesthetic.

### 1. Clinical and Pre-Clinical Anti-SARS-COV-2 Agents

In some forms, the additional therapeutic agent is or includes one or more agents that have been identified as effective for the treatment or prevention of coronavirus infection and/or diseases and disorders associated with coronavirus infections in a subject in need thereof. The agents can be FDA approved for clinical use, or pre-clinical agents, that are for example, undergoing clinical trials, for example, as described in Zumla, et al., Nat Rev Drug Discov 15, 327-347 (2016), the contents of which are incorporated herein in their entirety.

Exemplary additional anti-SARS-COV-2 agents effective for treating and/or preventing one or more symptoms of COVID. In some forms, the additional therapeutic agent is or includes one or more agents that have been identified as effective for the treatment or prevention of SARS-COV-2 infection and/or coronavirus disease (COVID) in a subject in need thereof.

Exemplary COVID-specific additional agents that can be administered in combination with the described antiviral PAP pathway inhibitors include Nirmatrelvir-Ritonavir (PAXLOVID), Remdesivir (VEKLURY), Molnupiravir (LAGEVIRIO), Ribavirin, alone or in combination with lopinavir-ritonavir, optionally further in combination with one or more corticosteroids, such as pulsed methylprednisolone; Interferon alfa-1 in combination with one or more corticosteroids; Convalescent-phase plasma; Ribavirin in combination with interferon alfa-2a and/or interferon alfa-2b; Mycophenolic acid, antiviral small molecule agent K22; dsRNA-activated caspase oligomerizer (DRACO) protein; SARS-COV PLpro inhibitors, such as GRL0617 and/or compound 4; nucleoside analogues, such as BCX4430; and Bananins and 5-hydroxychromone derivatives.

i. COVID Vaccines

In other forms, the additional therapeutic agent(s) include(s) one or more COVID vaccines, such as a vaccine developed and/or marketed by Pfizer-BioNTech or Moderna COVID-19 vaccine, and Novavax protein subunit COVID-19 vaccine, and Johnson & Johnson's Janssen (J&J/Janssen) viral vector COVID-19 vaccine.

COVID vaccines are known in the art. Any of the COVID vaccines can be administered in combination with the disclosed antiviral PAP pathway inhibitors. In some forms, the vaccine is an mRNA vaccine. Exemplary COVID vaccines include Pfizer-BioNTech or Moderna COVID-19 vaccine mRNA vaccines. Exemplary antigens encoded or encapsulated within the vaccines include SARS-COV-2 recombinant spike protein.

In exemplary forms, the disclosed antiviral PAP pathway inhibitors are administered as a prophylactic agent to prevent potential coronavirus infections and/or reduce the likelihood of spreading a coronavirus infection. Therefore, in some forms, the disclosed antiviral PAP pathway inhibitor(s) is administered as a prophylactic agent in combination with one or more COVID vaccines. In some forms, the subject has previously been vaccinated with one or more COVID vaccines. In some forms, the subject who has previously received one or more COVID vaccines is administered another dose of the same or a different COVID vaccine, for example, a “booster” COVID vaccine, together with the disclosed antiviral PAP pathway inhibitor(s).

ii. Anti-Coronavirus Combined Drug Strategies

In some forms, the disclosed antiviral PAP pathway inhibitors are combined with one or more additional antiviral drugs that has a desired antiviral activity to enhance the breadth of cross-reactivity, and/or efficacy of the antiviral effect relative to that of the PAP pathway inhibitor administered on it own. For example, in some forms, the combination of antiviral PAP pathway inhibitors provides an enhanced antiviral effect against an infecting coronavirus in a subject. In some forms, the compositions of antiviral PAP pathway inhibitors combine the antiviral efficacy of two or more agents that are active against the same or different coronavirus strains. In some forms, an antiviral PAP pathway inhibitor (i.e., that targets the host cell) is combined with an agent that targets the infectious virus, to provide enhanced antiviral activity and/or broader virus-type specificity and/or to overcome mutation-based viral resistance. In exemplary forms, the compositions include one or more of the disclosed antiviral PAP pathway inhibitors, together with an antiviral agent that directly block, inhibits or otherwise diminishes the activity of one or more viral surface receptors, or viral enzymes, or viral coat proteins, to reduce or prevent viral replication and/or production of viable virions in the host.

In some forms, an antiviral PAP pathway inhibitor, such as propranolol, is administered as a mixture in combination with Nirmatrelvir-Ritonavir (PAXLOVID). In some forms, an antiviral PAP pathway inhibitor, such as propranolol, is administered as a mixture in combination with Remdesivir (VEKLURY). In some forms, an antiviral PAP pathway inhibitor, such as propranolol, is administered as a mixture in combination with Molnupiravir (LAGEVIRIO). In some forms, an antiviral PAP pathway inhibitor, such as propranolol, is administered as a mixture in combination with Ribavirin, alone or in combination with lopinavir-ritonavir, optionally further in combination with one or more corticosteroids, such as pulsed methylprednisolone. In some forms, an antiviral PAP pathway inhibitor, such as propranolol, is administered as a mixture in combination with interferon alfa-1 in combination with one or more corticosteroids. In some forms, an antiviral PAP pathway inhibitor, such as propranolol, is administered as a mixture in combination with Convalescent-phase plasma. In some forms, an antiviral PAP pathway inhibitor, such as propranolol, is administered as a mixture in combination with Ribavirin in combination with interferon alfa-2a and/or interferon alfa-2b. In some forms, an antiviral PAP pathway inhibitor, such as propranolol, is administered as a mixture in combination with Mycophenolic acid. In some forms, an antiviral PAP pathway inhibitor, such as propranolol, is administered as a mixture in combination with antiviral small molecule agent K22. In some forms, an antiviral PAP pathway inhibitor, such as propranolol, is administered as a mixture in combination with SARS-COV PLpro inhibitors, such as GRL0617 and/or compound 4. In some forms, an antiviral PAP pathway inhibitor, such as propranolol, is administered as a mixture in combination with nucleoside analogues, such as BCX4430.

iii. Bronchodilators

In some forms, formulations of anti-coronavirus agent(s) that acts via the host PAP pathway, such as propranolol, additionally include one or more bronchodilators. Bronchodilators are a type of medication that helps open the airways to make breathing easier.

Short-acting bronchodilators in an emergency or as needed for quick relief. Some exemplary short-acting bronchodilators include anticholinergics such as ipratropium (e.g., ATROVENT®, in COMBIVENT®, in DUONEB®), beta2-agonists such as albuterol (e.g., VOSPIRE ER®, in COMBIVENT®, in DUONEB®), and levalbuterol (e.g., XOPENEX®).

Long-acting bronchodilators are used to treat COPD over an extended period of time. They are usually taken once or twice daily over a long period of time, and they come as formulations for inhalers or nebulizers. Some exemplary long-acting bronchodilators include anticholinergics such as aclidinium (e.g., TUDORZA®), tiotropium (e.g., SPIRIVA®), or umeclidinium (e.g., INCRUSE ELLIPTA®), beta2-agonists such as arformoterol (e.g., BROVANA®), formoterol (e.g., FORADIL®, PERFOROMIST®), indacaterol (e.g., ARCAPTA®), salmeterol (e.g., SEREVENT®), and olodaterol (e.g., STRIVERDI RESPIMAT®).

iv. Corticosteroids

In some forms, formulations of anti-coronavirus agent(s) that acts via the host PAP pathway, such as propranolol, additionally include one or more corticosteroids. Corticosteroids help reduce inflammation in the body, making air flow easier to the lungs. There are several corticosteroids. Some are prescribed with bronchodilators because these two medications can work together to make breathing more effective. Fluticasone (e.g., FLOVENT®), budesonide (e.g., PULMICORT®), and prednisolone are the ones doctors commonly prescribe for COPD.

v. Methylxanthines

In some forms, formulations of anti-coronavirus agent(s) that acts via the host PAP pathway, such as propranolol, additionally include one or more methylxanthine. Methylxanthines are heterocyclic compounds that are methylated derivatives of xanthine including coupled pyrimidinedione and imidazole rings (Talik et al., Separ. Purif. Rev. 2012; 41:1-61). Methylxanthines have been widely used for therapeutic purposes for decades, with proven therapeutic benefits in different medical scopes. For example, the naturally occurring methylxanthines like caffeine, theophylline, and theobromine have been used in the treatment of respiratory diseases (Lam and Newhouse, Chest. 1990; 98:44-52), cardiovascular diseases, cancer (Hayashi et al., Anticancer Res. 2005; 25:2399-2405; Kimura et al., J. Orthop. Sci. 2009; 14:556-565) and the commercially produced xanthine derivative drug like pentoxifylline has been widely documented to have immunomodulatory properties.

In some forms, formulations of anti-coronavirus agent(s) that acts via the host PAP pathway, such as propranolol, include pentoxifylline and/or caffeine. Potential beneficial properties of methylxanthines like pentoxifylline and caffeine as an adjuvant therapy to treat COVID-19 patients have been suggested (Monji F et al., Eur J Pharmacol. 2020 Nov. 15; 887:173561). In these cases, theophylline (e.g., THEO-24®, THEOLAIR®, ELIXOPHYLLINE®, QUIBRON-T®, UNIPHYL®, and ELIXOPHYLLIN®), can be used, which works as an anti-inflammatory and/or antioxidant, and relaxes the muscles in the airway, to take along with a bronchodilator. Theophylline comes as a pill or a liquid to be taken on a daily basis, and/or combined with other medications.

vi. Phosphodiesterase-4 Inhibitors

In some forms, formulations of anti-coronavirus agent(s) that acts via the host PAP pathway, such as propranolol, additionally include a Phosphodiesterase-4 such as roflumilast. Roflumilast is a PDE-4 inhibitor used as a comprehensive support for COVID-19 pathogenesis (Sugin Lal Jabaris S et al., Pulm Pharmacol Ther. 2021 February; 66: 101978). Roflumilast, a well-known anti-inflammatory and immunomodulatory drug, is protective against respiratory models of chemical and smoke induced lung damage. There is significant data which demonstrate the protective effect of PDE-4 inhibitor in respiratory viral models and is likely to be beneficial in combating COVID-19 pathogenesis.

In some forms, formulations of anti-coronavirus agent(s) that acts via the host PAP pathway, such as propranolol, include phosphodiesterase-4 inhibitors. In some forms, the compositions help relieve inflammation and/or improve air flow to the lungs. Several PDE-4 inhibitors have been identified such as cilomilast, piclamilast, oglemilast, tetomilast, tofimilast, ronomilast, revamilast, UK-500,001, AWD 12-281, CDP840, CI-1018, GSK256066, YM976, GS-5759 to treat chronic obstructive pulmonary disease (COPD) and asthma. CHF 6001, is an inhaled PDE-4 inhibitor currently undergoing phase II clinical trials for COPD. Also, two orally administered PDE-4 inhibitors such as roflumilast and apremilast have been approved in a row as treatments against inflammatory diseases including COPD, psoriasis, and psoriatic arthritis.

vii. Antimicrobial Agents

In some forms, formulations of anti-coronavirus agent(s) that acts via the host PAP pathway, such as propranolol, additionally include one or more antimicrobial agents. An antimicrobial agent is a substance that kills or inhibits the growth of microbes such as bacteria, fungi, viruses, or parasites. Antimicrobial agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines.

Other exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

viii. Local Anesthetics

In some forms, formulations of anti-coronavirus agent(s) that acts via the host PAP pathway, such as propranolol, additionally include one or more local anesthetics. A local anesthetic is a substance that causes reversible local anesthesia and has the effect of loss of the sensation of pain. Non-limiting examples of local anesthetics include ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof. In other aspects of this form, the formulations of anti-coronavirus agent(s) that acts via the host PAP pathway, such as propranolol, include an anesthetic agent in an amount of, e.g., about 10 mg, about 50 mg, about 100 mg, about 200 mg, or more than 200 mg. The concentration of local anesthetics in the compositions can be therapeutically effective meaning the concentration is adequate to provide a therapeutic benefit without inflicting harm to the patient.

ix. Anti-inflammatory Agents

In some forms, formulations of anti-coronavirus agent(s) that acts via the host PAP pathway, such as propranolol, additionally include one or more anti-inflammatory agents. Anti-inflammatory agents reduce inflammation and include steroidal and non-steroidal drugs. Suitable steroidal active agents include glucocorticoids, progestins, mineralocorticoids, and corticosteroids. Other exemplary anti-inflammatory agents include triamcinolone acetonide, fluocinolone acetonide, prednisolone, dexamethasone, loteprendol, fluorometholone, ibuprofen, aspirin, and naproxen. Exemplary immune-modulating drugs include cyclosporine, tacrolimus, and rapamycin. Exemplary non-steroidal anti-inflammatory drugs (NSAIDs) include mefenamic acid, aspirin, diflunisal, salsalate, ibuprofen, naproxen, fenoprofen, ketoprofen, deacketoprofen, flurbiprofen, oxaprozin, loxoprofen, indomethacin, sulindac, etodolac, ketorolac, diclofenac, nabumetone, piroxicam, meloxicam, tenoxicam, droxicam, lornoxicam, isoxicam, meclofenamic acid, flufenamic acid, tolfenamic acid, elecoxib, rofecoxib, valdecoxib, parecoxib, lumiracoxib, etoricoxib, firocoxib, sulphonanilides, nimesulide, niflumic acid, and licofelone. In some forms, the anti-inflammatory agents are anti-inflammatory cytokines. Exemplary cytokines are IL-10, TGF-β and IL-35.

### C. Formulations

Formulations of anti-coronavirus agent(s) that acts via the host PAP pathway, such as propranolol, are provided. The anti-coronavirus agent(s), such as propranolol, can be formulated for administration to a subject, for example, as a pharmaceutical formulation. Exemplary formulations include a solution, a dry powder, a tablet, micelles, colloids, nanodroplets, nano-structured hydrogel, nanocrystals, and a nanosuspension. Typically, the formulation includes a determined amount of anti-coronavirus agent(s), such as propranolol, in a form appropriate for a desired route of administration. The compositions can be stored lyophilized in single use vials for rehydration immediately before use. Other means for rehydration and administration are known to those skilled in the art.

The pharmaceutical formulations typically contain anti-coronavirus agent(s), such as propranolol, in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS) and may be administered to an individual without causing undesirable biological side effects or unwanted interactions.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704.

Exemplary formulations of anti-coronavirus agent(s), such as propranolol, include liquids and dry powders. In some forms, the antiviral peptides in an amount from about 1% to about 100%, inclusive, from about 1% to about 80%, from about 1% to about 50%, preferably from about 1% to about 40% by weight, more preferably from about 1% to about 20% by weight, most preferably from about 1% to about 10% by weight. The ranges above are inclusive of all values from 1% to 100%.

### 1. Dry Formulations

In some forms, anti-coronavirus agent(s), such as propranolol, are formulated in dry powder forms as finely divided solid formulations. The dry powder components can be stored in separate containers or mixed at specific ratios and stored. In some forms, suitable aqueous and organic solvents are included in additional containers. In other forms, dry powder components, one or more solvents, and instructions on procedures to mix and prepare assembled nanostructures are included in a kit. Alternatively, stabilized, assembled particles, nanoparticles or bulk gel thereof are dried via vacuum-drying or freeze-drying, and suitable pharmaceutical liquid carrier can be added to rehydrate and suspend the assembled nanostructures or gel compositions upon use.

Dry powder formulations are typically prepared by blending one or more gelators, stabilizing agents, or active agents with one or more pharmaceutically acceptable carriers. Pharmaceutical carrier may include one or more dispersing agents. The pharmaceutical carrier may also include one or more pH adjusters or buffers. Suitable buffers include organic salts prepared from organic acids and bases, such as sodium citrate or sodium ascorbate. The pharmaceutical carrier may also include one or more salts, such as sodium chloride or potassium chloride. The dry powder formulations can be suspended in the liquid formulations to form nanoparticle solutions and administered systemically or regionally using methods known in the art for the delivery of liquid formulations.

### 2. Liquid Formulations

In some forms, the anti-coronavirus agent(s), such as propranolol, are formulated as a liquid. Suitable liquid carriers include, but are not limited to, distilled water, de-ionized water, pure or ultrapure water, saline, and other physiologically acceptable aqueous solutions containing salts and/or buffers, such as phosphate buffered saline (PBS), Ringer's solution, and isotonic sodium chloride, or any other aqueous solution acceptable for administration to an animal or human.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

Formulations may be prepared using one or more pharmaceutically acceptable excipients, including diluents, preservatives, binders, lubricants, disintegrators, swelling agents, fillers, stabilizers, and combinations thereof. Liquid formulations may also contain minor amounts of polymers, surfactants, or other excipients well known to those of the art. In this context, “minor amounts” means no excipients are present that might adversely affect the delivery of the antiviral peptide compositions to organs or tissues, e.g., through circulation.

In some forms, the anti-coronavirus agent(s), such as propranolol, are formulated in a suitable carrier. A carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

In some forms, the anti-coronavirus agent(s), such as propranolol, are formulated to contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzyl peroxide, benzethonium chloride, benzyl alcohol, cetylpyridinium chloride, chlorobutanol, phenol, phenylethyl alcohol, and thimerosal.

In some forms, the anti-coronavirus agent(s), such as propranolol, are formulated to be buffered to a pH, for example, pH 2, 3, 4, 5, 6, 7, 8, 9 or pH 10. In an exemplary form, the formulation is typically buffered to a pH of 3-8 for parenteral administration. Suitable buffers include, but are not limited to, phosphate buffers, acetate buffers, and citrate buffers.

In some forms, the anti-coronavirus agent(s), such as propranolol, are formulated to include one or more water soluble polymers. Water soluble polymers are often used in formulations for parenteral administration. Suitable water-soluble polymers include, but are not limited to, polyvinylpyrrolidone, dextran, carboxymethylcellulose, and polyethylene glycol.

Generally, dispersions are prepared by incorporating the various sterilized gelators, stabilizing agents, and/or active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules.

### 3. Dosage Units

The anti-coronavirus agent(s), such as propranolol, compositions are preferably formulated in dosage unit form for case of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rats, rabbits, dogs, or pigs. The animal model is also used to achieve a desirable concentration range and route of administration. Such information should then be useful to determine useful doses and routes for administration in humans.

### III. Methods of Treatment

Methods for preventing or treating one or more symptoms of coronavirus infection in a subject in need thereof are described.

It has been established that reduction or inhibition of the host PAP pathway reduced or prevents replication of coronaviruses in the host. Therefore, methods of reducing coronavirus replication in a cell, including contacting a cell infected by a replicative coronavirus genome with an effective amount of an anti-coronavirus agent that inhibits one or more gene expression products associated with the phosphatidate phosphatase (PAP) pathway in the cell to reduce the replication of the coronavirus in the cell relative to an untreated control cell are provided.

In some forms, the anti-coronavirus agent is or includes propranolol, or a derivative or variant of propranolol. In some forms, the propranolol is at a concentration of between about 10 μM and 500 μM, inclusive. In some forms, the propranolol is at a concentration of about 100 μM. Typically, the cell is a mammalian cell, such as a human cell. In some forms, the contacting is in vitro. In other forms, the contacting is in vivo for example, in the body of a subject.

When the contacting is in vivo, the method includes administering to a subject in need thereof an effective amount of an anti-coronavirus agent that inhibits one or more gene expression products associated with the phosphatidate phosphatase (PAP) pathway in the subject to reduce the replication of the coronavirus in the subject relative to an untreated control subject. In some forms, the anti-coronavirus agent is or includes propranolol, or a derivative or variant of propranolol. For example, in some forms, propranolol is administered to the subject in an amount between about 0.01 mg/kg body weight of the subject and about 100 mg/kg body weight of the subject, inclusive. In an exemplary form, propranolol is administered in an amount between about 0.01 mg/kg body weight of the subject.

Typically, the methods administer an effective amount of anti-coronavirus agent(s) that acts via the host PAP pathway, or pharmaceutical formulations thereof to treat or prevent a disease, for example severe acute respiratory syndrome coronavirus 2 (SARS-CoV-2).

The anti-coronavirus agent(s) that acts via the host PAP pathway, may be administered to a subject in need thereof in any appropriate pharmaceutical carrier, such as a liquid, for example water, and saline, or a powder, for administration to the respiratory system.

As described in the Examples, an exemplary anti-coronavirus agent(s) that acts via the host PAP pathway is propranolol. Therefore, in some forms, the methods administer an effective amount of propranolol, or a derivative, or variant thereof that is effective as an anti-coronavirus agent, or pharmaceutical formulation thereof to treat or prevent a disease, for example severe acute respiratory syndrome coronavirus 2 (SARS-COV-2), in a subject in need thereof.

The propranolol, or a derivative, or variant thereof that is effective as an anti-coronavirus agent, or pharmaceutical formulation thereof, may be administered to a subject in need thereof in any appropriate pharmaceutical carrier, such as a liquid, for example water, and saline, or a powder, for administration to the respiratory system.

Typically, the methods administer an amount of anti-coronavirus agent(s) that acts via the host PAP pathway, such as propranolol, effective to reduce the replication of a coronavirus in a subject relative to a control, such as an untreated subject. In some forms, the methods reduce or prevent infection by the causative viral disease COVID-19 in a subject. In other forms, the methods prevent or reduces the invading viral pathogens in getting inside and/or proliferating in one or more targeting cells.

Generally, the formulations of anti-coronavirus agent(s) that acts via the host PAP pathway can be delivered by any method and/or device which is currently used for delivery to a subject in vivo.

In exemplary forms, methods for treating or preventing one or more symptoms of a coronavirus infection in a subject include administering to a subject in need thereof an effective amount of an anti-coronavirus agent that inhibits one or more gene expression products associated with the phosphatidate phosphatase (PAP) pathway in the subject to reduce the replication of the coronavirus in the subject relative to an untreated control subject. In some forms, the anti-coronavirus agent that inhibits one or more gene expression products associated with the phosphatidate phosphatase (PAP) pathway includes propranolol, or a derivative or variant of propranolol. In some forms, the propranolol is or includes only the L-isoform propranolol. In other forms, the propranolol is or includes only the D-isoform propranolol.

In some forms, the disclosed antiviral PAP pathway inhibitor(s), such as prpanolol, is administered to a subject as a prophylactic agent to prevent potential coronavirus infections and/or reduce the likelihood of spreading a coronavirus infection. In some forms, the disclosed antiviral PAP pathway inhibitor(s), such as prpanolol, is administered to a subject as a prophylactic agent to reduce the severity or duration of one or more symptoms in the event of a coronavirus infection. Therefore, in some forms, the disclosed antiviral PAP pathway inhibitor(s), such as prpanolol, is administered as a prophylactic agent to a subject who is not sick and/or is not infected with a cornavirus.

### A. Subjects to be Treated

Any of the described methods can include one or more steps of identifying a subject to be treated. A subject in need of treatment is a subject having or at risk of having an infection e.g., a subject having or at risk of contracting an infection with a coronavirus. The methods are particularly suited for those at risk of exposure to one or more respiratory pathogens such as SARS-COV-2. Thus, in some forms, the subject has not experienced any symptoms from COVID but is at risk of doing so.

A positive SARS-COV-2 viral test (i.e., reverse transcription polymerase chain reaction [RT-PCR] test or antigen test) or serologic (antibody) test can help assess for current or previous infection. In some forms, the methods retard the development of symptoms in a patient identified as positive via one or more tests for SARS-COV-2 viral infection, with or without any symptoms.

In some forms, the subject is an otherwise healthy subject. For example, in some forms, the subject does not have, or is not diagnosed as having a pre-existing or underlying disease or disorder or condition, such as cardiovascular disease. In some forms, the subject is not taking medication for any purpose other than for an infection, such as a viral infection. In some forms, the subject does not have, and/or has not been identified as having or at risk of having any one or more of Hypertension, Angina pectoris, Myocardial infarction, Tachycardia, Anxiety, panic attacks, hyperthyroidism, Portal hypertension, esophageal variceal bleeding and ascites, Post-traumatic stress disorder (PTSD) a, Aggressive behavior associated with brain injuries, Essential tremor, Migraine and/or cluster headache, Hyperhidrosis (excessive sweating), Infantile hemangioma Propranolol Glaucoma, Thyrotoxicosis, Hypertrophic cardiomyopathy or Atrial Fibrillation. Therefore, in some forms, the subject is not medicated for one or more of Hypertension, Angina pectoris, Myocardial infarction, Tachycardia, Anxiety, panic attacks, hyperthyroidism, Portal hypertension, esophageal variceal bleeding and ascites, Post-traumatic stress disorder (PTSD), Aggressive behavior associated with brain injuries, Essential tremor, Migraine and/or cluster headache, Hyperhidrosis (excessive sweating), Infantile hemangioma, Glaucoma, Thyrotoxicosis, Hypertrophic cardiomyopathy or Atrial Fibrillation. In other forms, the subject has or is diagnosed as having or at increased risk of having one or more of Hypertension, Angina pectoris, Myocardial infarction, Tachycardia, Anxiety, panic attacks, hyperthyroidism, Portal hypertension, esophageal variceal bleeding and ascites, Post-traumatic stress disorder (PTSD), Aggressive behavior associated with brain injuries, Essential tremor, Migraine and/or cluster headache, Hyperhidrosis (excessive sweating), Infantile hemangioma, Glaucoma, Thyrotoxicosis, Hypertrophic cardiomyopathy or Atrial Fibrillation.

In some forms, the subject is not and/or has never been administered a beta-adrenergic receptor blocking agent. In some forms, the subject is not and/or has never been administered Propranolol.

### B. Coronavirus Infections to be Treated

In some forms, the methods provide an effective amount of anti-coronavirus agent(s) that acts via the host PAP pathway, such as propranolol, to treat or prevent one or more symptoms of coronavirus infection in the subject, for example, reducing or preventing one or more symptoms or physiological markers of severe acquired respiratory syndrome (SARS) in a subject.

In some forms, the coronavirus includes one or more of the six human pathogenic coronaviruses, including one or more strain of the Severe acute respiratory syndrome coronavirus (SARS-COV), and/or one or more strains of the Middle East respiratory syndrome (MERS-COV), and/or one or more strains of the Human coronavirus 229E (HCoV-229E), and/or one or more strains of HCoV-NL63, and/or one or more strains of HCoV-OC43, and/or one or more strains of the HCoV-HKU1. Therefore, in some forms, the methods administer an effective amount of anti-coronavirus agent(s) that acts via the host PAP pathway, such as propranolol, to treat or prevent one or more symptoms of an infection of Severe acute respiratory syndrome coronavirus (SARS-COV) in a subject in need thereof. In some forms, the methods administer an effective amount of anti-coronavirus agent(s) that acts via the host PAP pathway, such as propranolol, to treat or prevent one or more symptoms of an infection of Middle East respiratory syndrome (MERS-COV) in a subject in need thereof. In some forms, the methods administer an effective amount of anti-coronavirus agent(s) that acts via the host PAP pathway, such as propranolol, to treat or prevent one or more symptoms of an infection of a Human coronavirus 229E (HCoV-229E) in a subject in need thereof. In some forms, the methods administer an effective amount of anti-coronavirus agent(s) that acts via the host PAP pathway, such as propranolol, to treat or prevent one or more symptoms of an infection of HCoV-NL63 in a subject in need thereof. In some forms, the methods administer an effective amount of anti-coronavirus agent(s) that acts via the host PAP pathway, such as propranolol, to treat or prevent one or more symptoms of an infection of HCoV-OC43 in a subject in need thereof. In some forms, the methods administer an effective amount of anti-coronavirus agent(s) that acts via the host PAP pathway, such as propranolol, to treat or prevent one or more symptoms of an infection of HCoV-HKU1 in a subject in need thereof.

Exemplary symptoms of COVID-19 include cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Therefore, in some forms, the methods prevent or reduce one or more of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

The coronavirus can be any one or more strains of a circulating or historic coronavirus. In some forms, the coronavirus (CoVs; subfamily Coronavirinae, family Coronaviridae, order Nidovirales) is an Alphacoronavirus, Betacoronavirus (BCoV), Gammacoronavirus or a Deltacoronavirus. In some forms, the Betacoronavirus is Severe Acute Respiratory Syndrome (SARS)-CoV-2 wild-type, SARS-COV-2 B.1.1.7 (Alpha variant), SARS-COV-2 B.1.351 (Beta variant), SARS-COV-2 P.1 (Gamma variant), SARS-COV-2 B.1.617, SARS-COV-2 B.1.617.1 (Kappa variant), SARS-COV-2 B.1.621 (Mu variant), SARS-COV-2 B.1.617.2 (Delta variant), SARS-COV-2 B.1.617.3, SARS-CoV-2 B.1.1.529 Omicron, SARS-COV-2 Omicron variant BA.1+R346K; SARS-COV-2 Omicron variant BA.2, SARS-COV-2 Omicron variant BA.3, SARS-COV-2 Omicron variant BA.4, and SARS-COV-2 Omicron variant BA.5.

### C. Methods of Administration

The compositions including anti-coronavirus agent(s) that acts via the host PAP pathway, such as propranolol, are generally administered to a subject in an effective amount for treating or preventing coronavirus infection in the subject.

As used herein the term “effective amount” means a dosage sufficient to inhibit, or prevent one or more infections, or symptoms of a coronavirus-related disease or to otherwise provide a desired pharmacologic and/or physiologic effect. The precise dosage will vary according to a variety of factors such as subject-dependent variables (e.g., age, immune system health, etc.), the specific variant of virus, and the treatment being affected.

The pharmaceutical compositions including one or more anti-coronavirus agent(s) that acts via the host PAP pathway, such as propranolol, can be for administration by parenteral (intramuscular, intraperitoneal, intravenous, or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In preferred forms, the compositions are administered systemically, for example via the oral, rectal or the intravenous route. In other forms, the composition is administered locally. Typically, local administration causes an increased localized concentration of the compositions which is greater than that which can be achieved by systemic administration. In some forms, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can affect a sustained release of the particles to the immediate area of the implant.

### D. Treatment Regimens

A treatment regimen can include one or multiple administrations of the compositions including one or more anti-coronavirus agent(s) that acts via the host PAP pathway, such as propranolol, for achieving a desired physiological change, including administering to an animal, such as a mammal, especially a human being, an effective amount of the compositions to treat the disease or symptom thereof, or to produce the physiological change. In preferred forms, the desired physiological change is the reduction in the amount of syncytial formation and lung damage in the subject.

### 1. Dosage and Effective Amounts

A therapeutically effective amount of anti-coronavirus agent(s) that acts via the host PAP pathway, such as propranolol, used in the treatment of diseases and disorders associated with coronavirus infection are typically sufficient to reduce or alleviate one or more symptoms of the diseases and disorders associated with coronavirus infection.

Symptoms of diseases and disorders associated with coronavirus infection may be cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Accordingly, the amount of anti-coronavirus agent(s) that acts via the host PAP pathway, such as propranolol, can be effective to, for example, treat or prevent one or more symptoms of a coronavirus infection. Preferably the anti-coronavirus agent(s) that acts via the host PAP pathway, such as propranolol, are delivered topically to the mucosal surface of the lung. Preferably the anti-coronavirus agent(s) that acts via the host PAP pathway, such as propranolol, do not target or otherwise modulate other metabolic processes or metabolic products. In some forms, the anti-coronavirus agent(s) that acts via the host PAP pathway, such as propranolol, are administered in an effective amount to coronavirus infection, or one or more diseases or disorders associated with coronavirus infection in a subject at risk of exposure to SAR-Cov-2 virus.

i. Effective Amounts

In some forms, the disclosed anti-coronavirus agent(s) that acts via the host PAP pathway, such as propranolol, is administered in an amount and for a time effective to reduce viral load a coronavirus in the body of a subject as compared to an untreated control. The reduction can be by an amount such as log 2-log 3. In some forms, the disclosed anti-coronavirus agent(s) that acts via the host PAP pathway, such as propranolol, is administered in an amount and for a time effective to decrease coronavirus genome copies in the body of a subject as compared to an untreated control. The reduction can be by an amount such as log 2-log 3. In some forms, the disclosed anti-coronavirus agent(s) that acts via the host PAP pathway, such as propranolol, is administered in an amount and for a time effective to virus titer in the lungs of a subject, as compared to an untreated control. The reduction can be by an amount such as log 2-log 3.

The actual effective amounts of anti-coronavirus agent(s) that acts via the host PAP pathway, such as propranolol, can vary according to factors including the specific agents administered, the particular composition formulated, the mode of administration, and the age, weight, condition of the subject being treated, as well as the route of administration and the disease or disorder.

In some forms, the effective amount of anti-coronavirus agent(s) that acts via the host PAP pathway, such as propranolol, causes little or no killing of cells within the subject, and preferably little or no inhibition of metabolism in cells. It is particularly preferred that the composition does not dampen activities of immune cells.

In the most preferred forms, methods of using the anti-coronavirus agent(s) that acts via the host PAP pathway, such as propranolol lead to direct or indirect reduction in the replication of coronaviruses, increase in the quality of life of those suffering from the disease, decrease in the dose of other medications required to treat the disease, delaying the progression of the disease, and/or prolonging survival of individuals.

ii. Doses

In some forms, the disclosed anti-coronavirus agent(s) that acts via the host PAP pathway, such as propranolol, is administered in a dosage amount. For example, in particular forms, the subject is administered a dosage of between about 0.01 mg/kg body weight and 100 mg/kg body weight, inclusive, of anti-coronavirus agent(s) that acts via the host PAP pathway, such as propranolol. In some forms, the subject is administered a dosage of between about 0.001 mg/kg body weight and 1.0 mg/kg body weight, inclusive, of anti-coronavirus agent(s) that acts via the host PAP pathway, such as propranolol. In some forms, the subject is administered a dosage of between about 0.02 mg/kg body weight and 1.0 mg/kg body weight, inclusive, of anti-coronavirus agent(s) that acts via the host PAP pathway, such as propranolol. In some forms, the subject is administered a dosage of between about 0.03 mg/kg body weight and 1.0 mg/kg body weight, inclusive, of anti-coronavirus agent(s) that acts via the host PAP pathway, such as propranolol. In some forms, the subject is administered a dosage of between about 0.04 mg/kg body weight and 1.0 mg/kg body weight, inclusive, of anti-coronavirus agent(s) that acts via the host PAP pathway, such as propranolol.

In some forms, the subject is administered a dosage of between about 0.01 mg/kg body weight and 1.0 mg/kg body weight, inclusive, of propranolol. In some forms, the subject is administered a dosage of between about 0.1 mg/kg body weight and 10 mg/kg body weight, inclusive, of propranolol. In some forms, the subject is administered a dosage of between about 1 mg/kg body weight and 40 mg/kg body weight, inclusive, of propranolol. In some forms, the subject is administered a dosage of between about 10 mg/kg body weight and 100 mg/kg body weight, inclusive, of propranolol.

In some forms, the anti-coronavirus agent(s) that acts via the host PAP pathway is propranolol. Dosage forms and pharmaceutically acceptable amounts of propranolol for administration to a human in vivo are known. In some forms, formulated as an oral dosage form. In some forms, the oral dosage form is an aqueous solution. When the oral dosage form of propranolol is an aqueous solution, the amount administered for adults is an amount between 180 to 240 milligrams (mg) per day, inclusive. In some forms, the total daily dosage is administered in divided doses. In some forms, when the oral dosage form of propranolol is an aqueous solution, the amount administered is based on body weight and can be determined by a clinician.

In some forms, the oral dosage form is a powder or tablet a gel or liquid and a coating. When the oral dosage form of propranolol is a powder or tablet, or an oral capsule including a gel or liquid and a coating the amount administered for adults is about 40 milligrams (mg) three times a day, i.e., for a total daily dosage of about 120 mg, or 60 milligrams (mg) per day, or 30 mg per day, or 80 milligrams (mg) once a day. In some forms, the dose is not more than 320 mg per day.

In some forms, the subject is administered a dosage of between about 0.1 and 1.0 mg, inclusive, of propranolol in a day. In some forms, the subject is administered a dosage of between about 1 mg and 10 mg, inclusive, of propranolol in a day. In some forms, the subject is administered a dosage of between about 1 mg and 40 mg, inclusive, of propranolol in a day. In some forms, the subject is administered a dosage of between about 10 mg and 100 mg, inclusive, of propranolol in a day.

iii. Dosing Regimens

In some forms, dosages of anti-coronavirus agent(s) that acts via the host PAP pathway, such as propranolol, are administered once, twice, or three times daily, or every other day, two days, three days, four days, five days, or six days to a human. In some forms, dosages of antiviral peptides are administered about once or twice every week, every two weeks, every three weeks, or every four weeks. In some forms, dosages are administered about once or twice every month, every two months, every three months, every four months, every five months, or every six months.

In some forms, the regimen includes one or more cycles of a round of therapy with antiviral peptides followed by a drug holiday (e.g., no antiviral peptides). The round of the therapy can be, for example, any of the administrations discussed above. Likewise, the drug holiday can be 1, 2, 3, 4, 5, 6, or 7 days; or 1, 2, 3, 4 weeks, or 1, 2, 3, 4, 5, or 6 months. Particular dosage regimens include, for example, one or more cycles in which the subject is administered the anti-coronavirus agent(s) that acts via the host PAP pathway, such as propranolol on each of two, three, four, five, six or seven days, weeks or months in a row, followed by a one, two, three, four, five, six or seven-day, week, or month drug holiday.

In an exemplary form, methods of treating or preventing one or more symptoms of COVID in a subject, include administering to a subject in need thereof an effective amount of propanolol, or a derivative or analog thereof, to prevent or reduce one or more symptoms of COVID in the subject relative to an untreated control subject, whereby the subject does not have a disease or disorder selected from hypertension, angina pectoris, myocardial infarction, tachycardia, anxiety, panic attacks, hyperthyroidism, portal hypertension, esophageal variceal bleeding and ascites, post-traumatic stress disorder (PTSD), aggressive behavior associated with brain injuries, essential tremor, migraine and/or cluster headache, hyperhidrosis, infantile hemangioma, glaucoma, thyrotoxicosis, hypertrophic cardiomyopathy and atrial fibrillation, and whereby the propranolol is administered to the subject in an amount between about 0.1 mg/kg body weight of the subject and about 40 mg/kg body weight of the subject, inclusive. In some forms, the propranolol is administered daily for a period of three days, or more than three days.

### 2. Controls

The effect of the compositions including anti-coronavirus agent(s) that acts via the host PAP pathway, such as propranolol can be compared to a control. Suitable controls are known in the art and include, for example, an untreated subject, or a placebo-treated subject.

A typical control is a comparison of a condition or symptom of a subject prior to and after administration of the targeted agent. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some forms, the symptom, pharmacologic, or physiologic indicator is measured in a subject prior to treatment, and again one or more times after treatment is initiated. In some forms, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

### E. Combination Therapies and Procedures

The described compositions of anti-coronavirus agent(s) that acts via the host PAP pathway, such as propranolol, can be administered alone or in combination with one or more conventional therapies.

In some forms, the conventional therapy includes administration of one or more of the compositions in combination with one or more additional active agents. The combination therapies can include administration of the active agents together in the same admixture, or in separate admixtures. Therefore, in some forms, the pharmaceutical composition includes two, three, or more active agents. Such formulations typically include an effective amount of an agent targeting the site of treatment. The additional active agent(s) can have the same or different mechanisms of action. In some forms, the combination results in an additive effect on the treatment of the lung condition. In some forms, the combinations result in a more than additive effect on the treatment of the disease or disorder.

The additional therapy or procedure can be simultaneous or sequential with the administration of the composition. In some forms, the additional therapy is performed between drug cycles or during a drug holiday that is part of the dosage regime. For example, in some forms, the additional therapy or procedure is damage control surgery, fluid resuscitation, blood transfusion, bronchoscopy, and/or drainage.

In some forms, the formulation including anti-coronavirus agent(s) that acts via the host PAP pathway, such as propranolol, is used in combination with oxygen therapy. In further forms, the additional therapy or procedure is prone positioning, recruitment maneuver, inhalation of NO, extracorporeal membrane oxygenation (ECMO), intubation, and/or inhalation of PGI₂. A prone position enhances lung recruitment in a potentially recruitable lung by various mechanisms, releasing the diaphragm, decreasing the effect of heart and lung weight and shape on lung tissue, decreasing the lung compression by the abdomen, and releasing the lower lobes, which improves gas exchange and decreases mortality in severe ARDS patients. ECMO provides extracorporeal gas exchange with no effect on lung recruitment. It affords lung rest and works well for the non-recruitable lung. It has been shown to improve survival for certain groups of patients in high-performance ECMO centers. Additional therapeutic agents can also include one or more of antibiotics, surfactant, corticosteroids, and glucocorticoids.

In some forms, the compositions and methods are used prior to or in conjunction, subsequent to, or in alternation with treatment with one or more additional therapies or procedures.

### IV. Kits

Kits are also disclosed. The kit can include a single dose or a plurality of doses of a composition including one or more anti-coronavirus agent(s) that acts via the host PAP pathway, such as propranolol, or pharmaceutical formulation thereof, and instructions for administering the compositions.

Specifically, the instructions direct that an effective amount of the composition including anti-coronavirus agent(s) that acts via the host PAP pathway, such as propranolol, be administered to an individual at risk of exposure to one or more coronaviruses such as a SARS-COV2 virus. The composition can be formulated as described above with reference to a particular treatment method and can be packaged in any convenient manner.

The present invention will be further understood by reference to the following non-limiting paragraphs.
1. A method of reducing coronavirus replication in a cell, the method including contacting a cell including a replicative coronavirus genome with an effective amount of an anti-coronavirus agent that inhibits one or more gene expression products associated with the phosphatidate phosphatase (PAP) pathway in the cell to reduce the replication of the coronavirus in the cell relative to an untreated control cell.
2. The method of paragraph 1, wherein the anti-coronavirus agent that inhibits one or more gene expression products associated with the phosphatidate phosphatase (PAP) pathway includes propranolol, or a derivative or variant of propranolol.
3. The method of paragraph 2, wherein the propranolol includes L-isoform propranolol.
4. The method of paragraph 2, wherein the propranolol includes D-isoform propranolol.
5. The method of paragraph 2, wherein the propranolol includes L-isoform propranolol and D-form propranolol.
6. The method of any one of paragraphs 2-5, wherein the propranolol is at a concentration of between about 10 μM and 500 μM, inclusive, optionally wherein the propranolol is at a concentration of about 100 μM.
7. The method of any one of paragraphs 1-6, wherein the cell is a human cell.
8. The method of any one of paragraphs 1-7, wherein the contacting is in vivo.
9. The method of any one of paragraphs 1-8, wherein the coronavirus is selected from the group including Severe Acute Respiratory Syndrome (SARS)-CoV-2 wild-type, SARS-COV-2 Alpha variant B.1.1.7, SARS-COV-2 Beta variant B.1.351, SARS-COV-2 Gamma variant P.1, SARS-COV-2 B.1.617, SARS-COV-2 Kappa variant B.1.617.1, SARS-COV-2 Mu variant B.1.621, SARS-COV-2 Delta variant B.1.617.2, SARS-COV-2 B.1.617.3, SARS-COV-2 Omicron variant B.1.1.529, SARS-COV-2 Omicron variant BA.1+R346K, SARS-COV-2 Omicron variant BA.2, SARS-COV-2 Omicron variant BA.3, SARS-COV-2 Omicron variant BA.4, SARS-COV-2 Omicron variant BA.5, and Middle

East respiratory syndrome (MERS-COV).
10. A method of treating or preventing one or more symptoms of a coronavirus infection in a subject, the method including
administering to a subject in need thereof an effective amount of an anti-coronavirus agent that inhibits one or more gene expression products associated with the phosphatidate phosphatase (PAP) pathway in the subject to reduce the replication of the coronavirus in the subject relative to an untreated control subject.
11. The method of paragraph 10, wherein the anti-coronavirus agent that inhibits one or more gene expression products associated with the phosphatidate phosphatase (PAP) pathway includes propranolol, or a derivative or variant of propranolol.
12. The method of paragraph 11, wherein the propranolol includes L-isoform propranolol.
13. The method of paragraph 11, wherein the propranolol includes D-isoform propranolol.
14. The method of paragraph 11, wherein the propranolol includes L-isoform propranolol and D-form propranolol.
15. The method of any one of paragraphs 11-14, wherein the propranolol is administered to the subject in an amount between about 0.01 mg/kg body weight of the subject and about 100 mg/kg body weight of the subject, inclusive,
optionally wherein the propranolol is in an amount between about 0.01 mg/kg body weight of the subject.
16. The method of any one of paragraphs 11-15, wherein the subject does not have a disease or disorder selected from the group including hypertension, angina pectoris, myocardial infarction, tachycardia, anxiety, panic attacks, hyperthyroidism, portal hypertension, esophageal variceal bleeding and ascites, post-traumatic stress disorder (PTSD), aggressive behavior associated with brain injuries, essential tremor, migraine and/or cluster headache, hyperhidrosis, infantile hemangioma, glaucoma, thyrotoxicosis, hypertrophic cardiomyopathy and atrial fibrillation.
17. The method of any one of paragraphs 11-16, wherein the subject does not have a coronavirus infection.
18. The method of any one of paragraphs 11-16, wherein the coronavirus is selected from the group including Severe Acute Respiratory Syndrome (SARS)-CoV-2 wild-type, SARS-COV-2 Alpha variant B.1.1.7, SARS-COV-2 Beta variant B.1.351, SARS-COV-2 Gamma variant P.1, SARS-COV-2 B.1.617, SARS-COV-2 Kappa variant B.1.617.1, SARS-COV-2 Mu variant B.1.621, SARS-COV-2 Delta variant B.1.617.2, SARS-COV-2 B.1.617.3, SARS-COV-2 Omicron variant B.1.1.529, SARS-COV-2 Omicron variant BA.1+R346K, SARS-COV-2 Omicron variant BA.2, SARS-COV-2 Omicron variant BA.3, SARS-COV-2 Omicron variant BA.4, SARS-COV-2 Omicron variant BA.5, and Middle East respiratory syndrome (MERS-COV).
19. The method of any one of paragraphs 1-18, wherein the one or more symptoms is selected from the group including cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.
20. The method of any one of paragraphs 11-19, wherein the composition is administered via the oral, rectal or intravenous route.
21. The method of any one of paragraphs 11-19, wherein the composition is administered in a form selected from the group including powder, liquids, and suspensions.
22. The method of any one of paragraphs 11-21, wherein the composition is administered in combination with another therapeutic, prophylactic, or diagnostic agent.
23. The method of any one of paragraphs 11-22, wherein the composition is administered in combination with one or more agents selected from the group including bronchodilators, corticosteroids, methylxanthines, phosphodiesterase-4 inhibitors, anti-angiogenesis agents, antimicrobial agents, antioxidants, anti-inflammatory agents, immunosuppressant agents, and anti-allergic agents, or combinations thereof.
24. The method of any one of paragraphs 11-23, wherein the composition is administered in combination with one or more agents selected from the group including Nirmatrelvir-Ritonavir (PAXLOVID), Remdesivir (VEKLURY), Molnupiravir (LAGEVIRIO), Ribavirin with lopinavir-ritonavir and one or more corticosteroids, Interferon alfa-1, Convalescent-phase plasma, Ribavirin in combination with interferon alfa-2a and/or interferon alfa-2b, Mycophenolic acid, antiviral small molecule agent K22; dsRNA-activated caspase oligomerizer (DRACO) protein, SARS-COV PLpro inhibitor GRL0617, nucleoside analogue BCX4430, and 5-hydroxychromone derivatives, or combinations thereof.
25. The method of any one of paragraphs 11-24, wherein the composition is administered more than once, at an interval selected from the group including once a day, twice a day, three times a day, once a week, twice a week, three times a week, once every two weeks, approximately once a month, once every two months and once every three months.
26. The method of any one of paragraphs 11-25, wherein the composition is administered once a day or more than once a day for up to a period of 1, 2, 3, 4, 5, or 6 weeks or months.
27. A method of treating or preventing one or more symptoms of COVID in a subject, the method including administering to a subject in need thereof an effective amount of propanolol, or a derivative or analog thereof, to prevent or reduce one or more symptoms of COVID in the subject relative to an untreated control subject.
28. The method of any one of paragraph 27, wherein the propranolol is administered to the subject in an amount between about 0.01 mg/kg body weight of the subject and about 100 mg/kg body weight of the subject, inclusive, optionally wherein the propranolol is in an amount between about 0.01 mg/kg body weight of the subject.
29. The method of any one of paragraphs 27-28, wherein the subject does not have a disease or disorder selected from the group including hypertension, angina pectoris, myocardial infarction, tachycardia, anxiety, panic attacks, hyperthyroidism, portal hypertension, esophageal variceal bleeding and ascites, post-traumatic stress disorder (PTSD), aggressive behavior associated with brain injuries, essential tremor, migraine and/or cluster headache, hyperhidrosis, infantile hemangioma, glaucoma, thyrotoxicosis, hypertrophic cardiomyopathy and atrial fibrillation.
30. The method of any one of paragraphs 27-29, wherein the one or more symptoms is selected from the group including cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.
31. The method of any one of paragraphs 27-30, wherein the COVID includes an infection by a virus selected from the group including Severe Acute Respiratory Syndrome (SARS)-CoV-2 wild-type, SARS-COV-2 Alpha variant B.1.1.7, SARS-COV-2 Beta variant B.1.351, SARS-COV-2 Gamma variant P.1, SARS-COV-2 B.1.617, SARS-COV-2 Kappa variant B.1.617.1, SARS-COV-2 Mu variant B.1.621, SARS-COV-2 Delta variant B.1.617.2, SARS-COV-2 B.1.617.3, SARS-COV-2 Omicron variant B.1.1.529, SARS-CoV-2 Omicron variant BA.1+R346K, SARS-COV-2 Omicron variant BA.2, SARS-COV-2 Omicron variant BA.3, SARS-COV-2 Omicron variant BA.4, SARS-COV-2 Omicron variant BA.5, and Middle East respiratory syndrome (MERS-COV).

The present invention will be further understood by reference to the following non-limiting examples.

### EXAMPLES

### Example 1: Inhibition of Phosphatidic Acid Phosphatase (PAP) Prevents Efficient SARS-COV-2 Replication

It was previously shown that host lipidomic perturbations are induced by SARS-CoV-2 infection in vitro and in vivo. Pathway analysis and downstream validation studies identified phosphatidic acid phosphatases (PAPs) as essential host factors for efficient SARS-COV-2 replication. Active agents that affect the host PAP enzyme inhibit the replication of multiple coronaviruses including SARS-COV-2 wild type, variants, and MERS-COV at the cell level.

### In Vitro Anti-SARS-COV-2 Effects of Propranolol

Propranolol was reported to inhibit both PAP-1 and PAP-2 even though PAP-2 exhibited no significant effect on SARS-COV-2 replication (Kumar, et al., Phosphatidic acid homeostasis regulated by a type-2 phosphatidic acid phosphatase represents a novel druggable target in malaria intervention. Cell Death Discov. 2019; 5:107; Fuentes, et al., Bromoenol lactone promotes cell death by a mechanism involving phosphatidate phosphohydrolase-1 rather than calcium-independent phospholipase A2. J Biol Chem. 2003; 278(45): 44683-90; and Yan, et al. Phosphatidic acid phosphatase 1 impairs SARS-CoV-2 replication by affecting the glycerophospholipid metabolism pathway. Int J Biol Sci. 2022; 18(12): 4744-55).

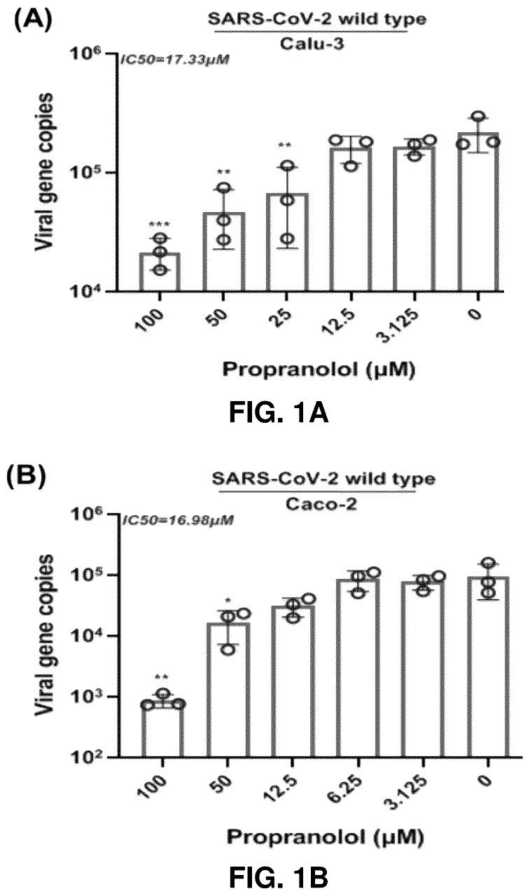

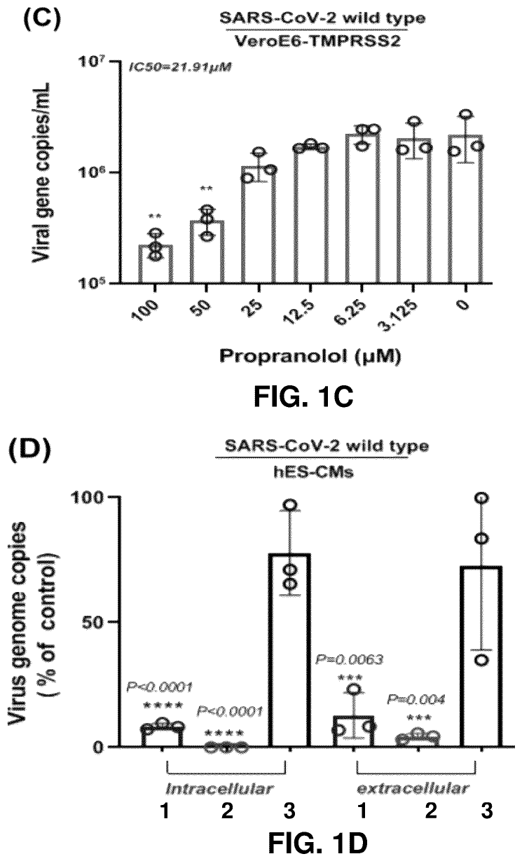

The results showed propranolol dose-dependently reduces SARS-COV-2 wild type gene copies in the culture supernatant at non-toxic concentrations in multiple cell lines such as Caco-2, Calu-3 and Vero-TMPRSS2 (FIG. 1A to 1C). To investigate if propranolol also works in other cell lines, the human embryonic stem cells-derived cardiomyocytes (hES-CMs), which is a good primary cell model susceptible to SARS-CoV-2 infection (Sharma, et al. Human iPSC-Derived Cardiomyocytes Are Susceptible to SARS-COV-2 Infection. Cell Rep Med. 2020; 1(4):100052), were applied to the current experiments. Indeed, both extracellular and intracellular SARS-COV-2 viral load remarkably diminished after propranolol treatment, which was in similar magnitude as that of remdesivir (FIG. 1D).

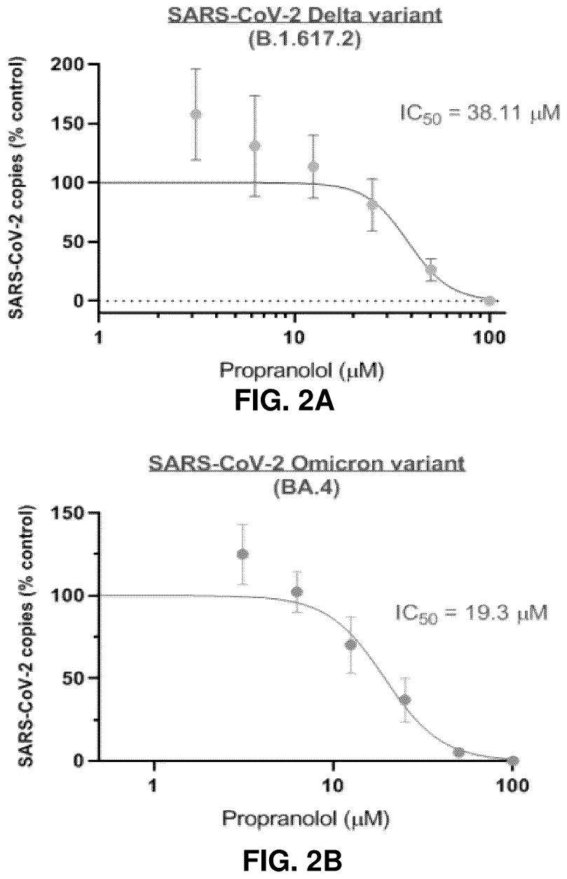

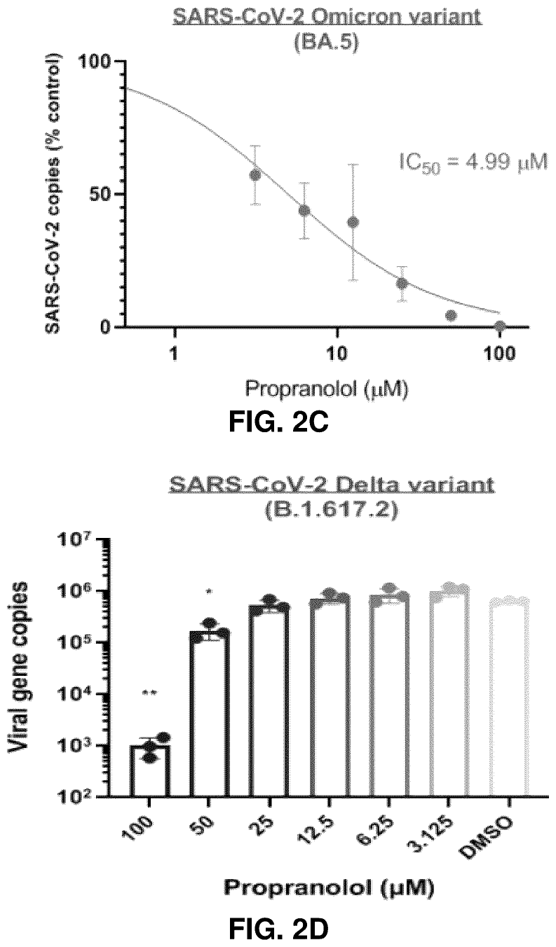

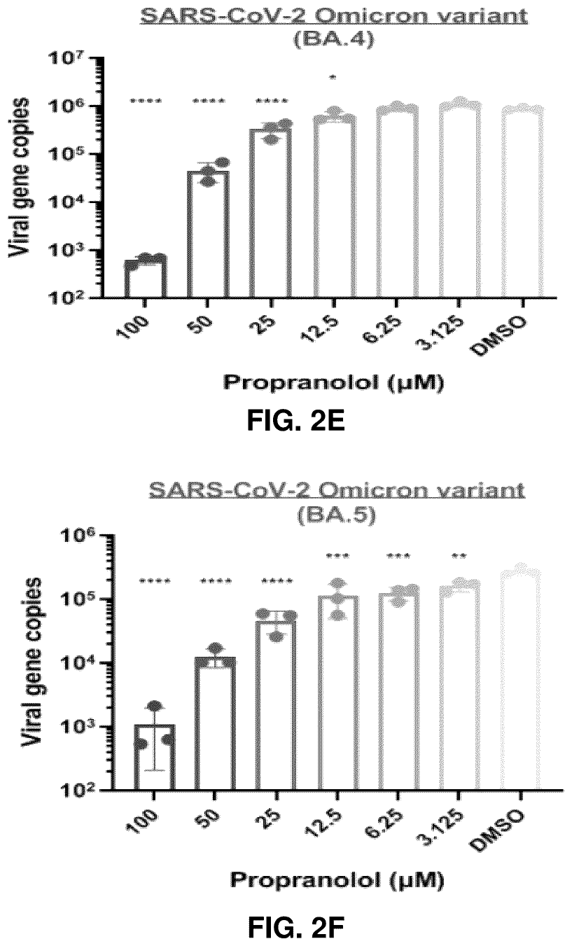

Importantly, it was showed that propranolol was also effective against the B.1.617.2 (Delta) (FIGS. 2A and 2D), emerging BA.4 (Omicron) (FIGS. 2B and 2E) and BA.5 (Omicron) (FIGS. 2C and 2F) variants with about 2-3 log₁₀ reduction in viral load at 100 μM of the drug. Taken together, these findings demonstrated propranolol inhibits wild-type and different variants of concern of SARS-COV-2 via inhibition of host PAP1/2 enzymes.

### In Vitro Antiviral Effects of Propranolol for Other Human-Pathogenic Coronaviruses

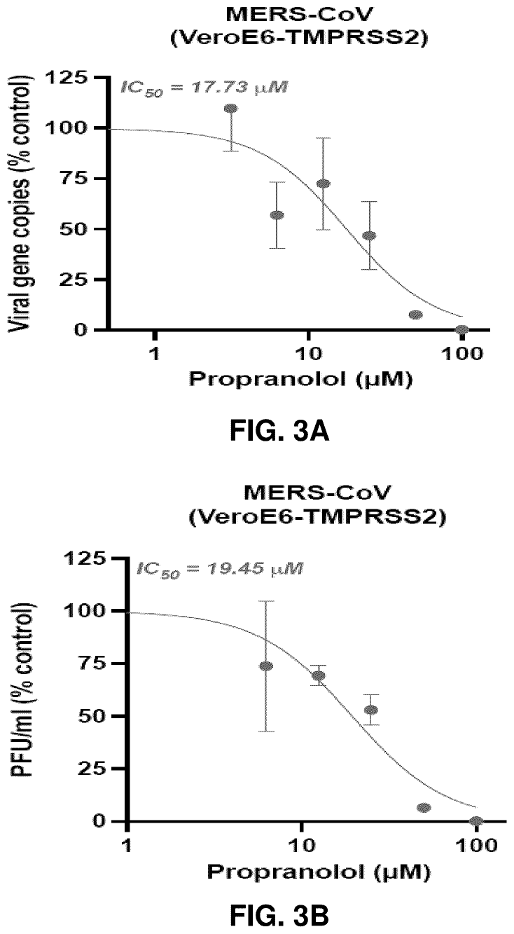

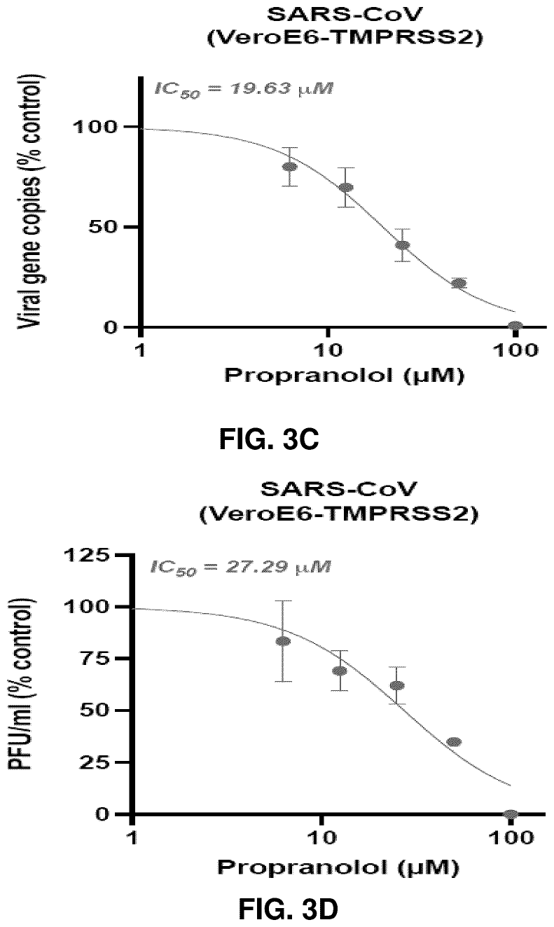

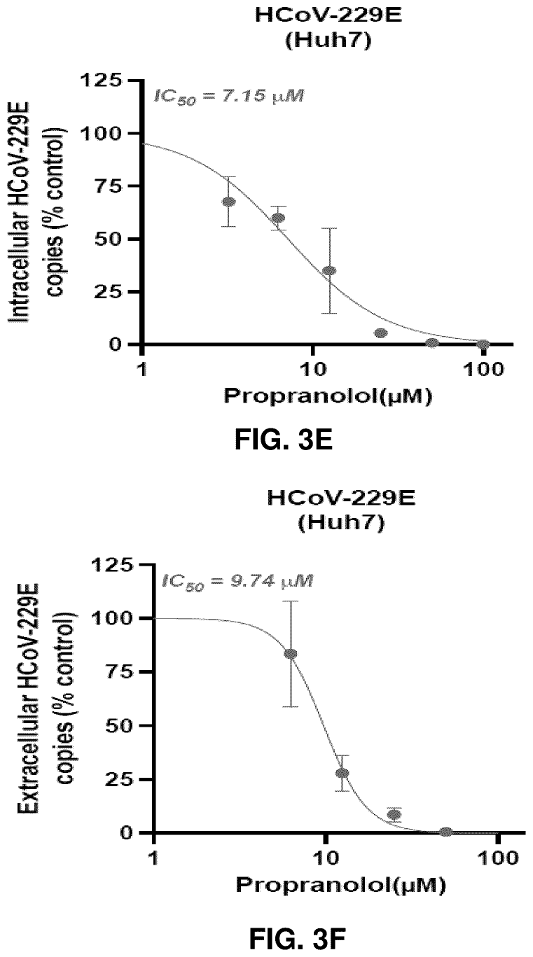

To further investigate if the antiviral effects of propranolol was conserved among other human-pathogenic coronaviruses, RT-qPCR and plaque assays were utilized to evaluate its antiviral efficacy for MERS-COV, SARS-COV and HCoV-229E in corresponding cell lines that support virus replication. The current results showed the IC50 of propranolol calculated by viral gene copies ranged from 17.73 μM for MERS-COV (FIG. 3A), 19.63 μM for SARS-COV (FIG. 3C) to 7.15 μM for HCoV-229E in the cell lysate (FIG. 3E) and 9.74 μM for HCoV-229E in the supernatant samples (FIG. 3F). In addition, the IC50 values of propranolol calculated using viral plaques suggests that propranolol effectively reduced the number of infectious viruses of MERS-COV (FIG. 3B) and SARS-COV (FIG. 3D).

### The Anti-SARS-COV-2 Effect Evaluation of Other Beta Antagonist or Agonists

Propranolol is a well-known beta blocker. To clearly clarify possible host factors relevant to the antiviral effect of propranolol, the beta-adrenergic receptor was explored as a host target that could affect the SARS-COV-2 replication.

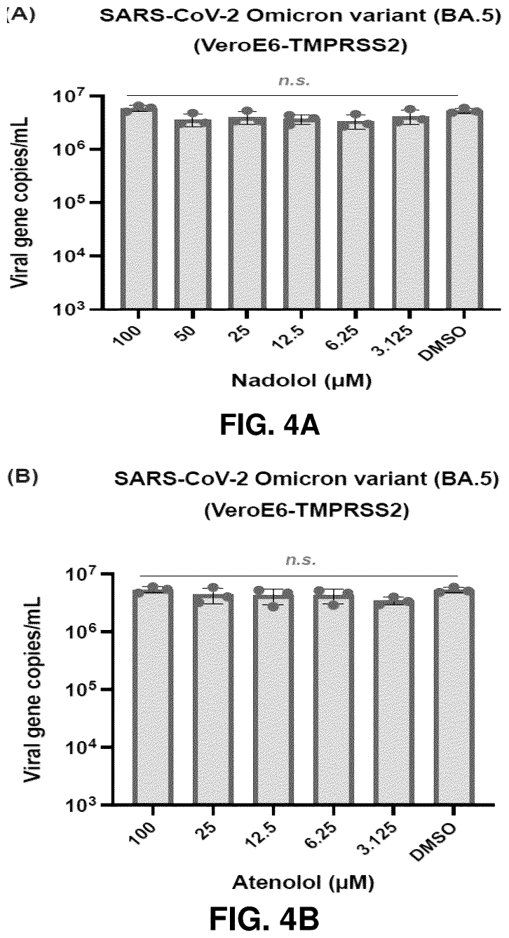

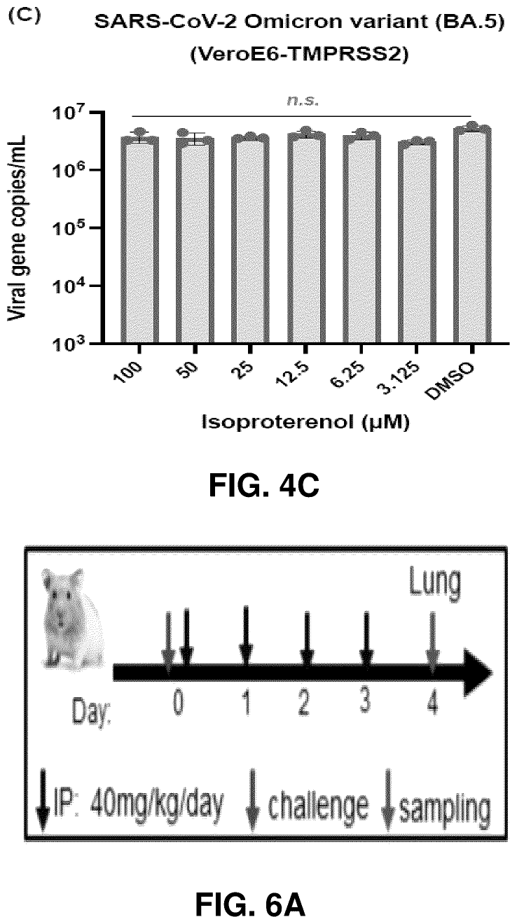

Nadolol (non-selective blocker), atenolol (beta 1 blocker), and isoproterenol (beta agonist) were applied to SARS-COV-2 infected cells for evaluating antiviral properties of beta-adrenergic receptors. Surprisingly, all tested compounds didn't affect the SARS-CoV-2 replication fitness no matter of beta antagonist or agonist at multiple dosages (FIGS. 4A, 4B and 4C).

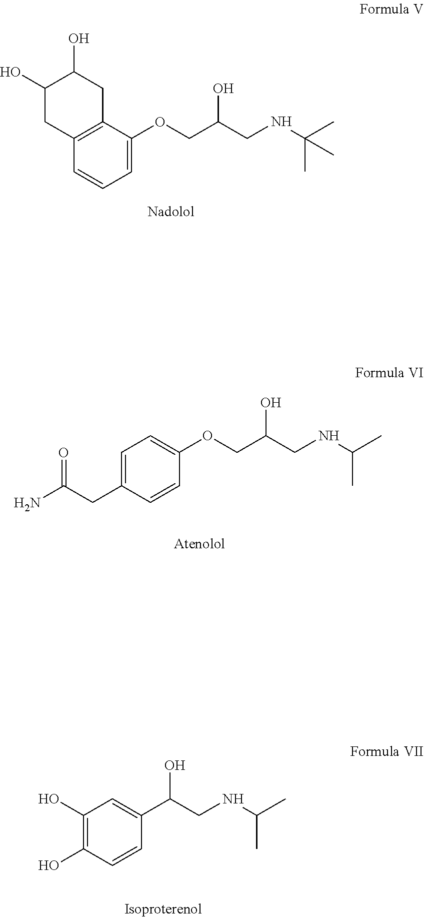 [SMILES: CC(C)(C)NCC(O)COc1cccc2c1CC(O)C(O)C2 ; CC(C)NCC(O)COc1ccc(CC(N)=O)cc1 ; CC(C)NCC(O)c1ccc(O)c(O)c1]

Collectively, the data indicate propranolol exerts a broad-spectrum anti-coronavirus efficacy via host PAP-1 enzyme inhibition (e.g., lipin3) rather than beta-adrenergic receptor as traditionally host target, which wasn't reported previously.

### Example 2: Lipins Co-Immunoprecipitate with SARS-COV2-NP

### Methods

Previous work showed a higher dependence of SARS-COV-2 on lipin3 than lipin2, and lipin1 was also reported to play an important role in the progression of various viral infections (Yan, et al., Int J Biol Sci. 2022; 18 (12): 4744 55; Castro, et al. Cells. 2019; 8 (11); Zhang, et al. PLOS Pathog, 2018; 14 (4): e 1006988; and Mingorance, et al. Plos Pathogens. 2018; 14 (9)). Moreover, the nucleocapsid protein (NP) stands out as the most abundantly expressed structural protein among all SARS-COV-2 viral proteins.

To further investigate the potential interaction between NP and lipins, lipin1 and lipin3 were selected as the representative PAP-1 enzymes to detect using a co-immunoprecipitation (co-ip) assay. A co-immunoprecipitation assay was conducted in HEK293T cells transfected with lipin1, lipin3, and NP plasmids. Protein A/G magnetic beads and monoclonal SARS-COV2-NP antibodies were employed to capture the target protein complex, which was then identified using anti-flag antibodies.

### Results

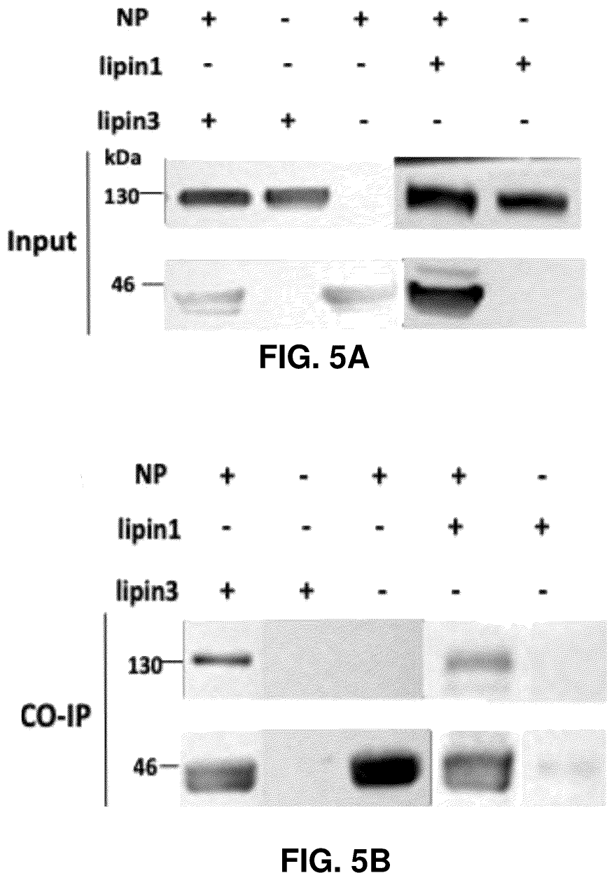

A co-immunoprecipitation (co-ip) assay revealed that either the lipin1 or lipin3 bands were observed as a pull-down product of NP (FIGS. 5A-5B), indicating an interaction between lipin1/lipin3 and SARS-COV-2 NP. Thus, propranolol demonstrates antiviral efficacy by modulating PAP-1 enzyme activity, thereby influencing coronavirus fitness.

### Example 3: Propranolol Ameliorates SARS-COV-2 Disease in a Hamster Model Methods

### Golden Syrian Hamster Model

Male and female Syrian hamsters, aged 6-10 weeks old, were kept in biosafety level housing and given access to standard pellet feed and water ad libitum. Hamsters were randomly allocated to experimental groups for antiviral evaluation. No blinding was applied.

To investigate the antiviral effects of propranolol against wild-type SARS-COV-2 (WT SARS-COV-2), hamsters were intranasally inoculated with 10⁴ PFU of WT SARS-CoV-2 in 100 μL PBS under intraperitoneal ketamine (200 mg/kg) and xylazine (10 mg/kg) anesthesia. The treatment regimen used intraperitoneal injection of propranolol that was given 6 hours post-infection (hpi) (the first therapeutic dose, 40 mg/kg/day) and the other 3 consecutive doses. Propranolol was delivered using PBS as a vehicle. To assess the regimen, animals were sacrificed at 4 days post-infection (dpi) for virological analyses. Lung tissue samples were collected for virology investigations. The viral gene copies in hamster lung tissues were determined by RT-qPCR and normalized with human β-actin (n=3); viral yields in the tissue homogenates were also detected by plaque assay.

Viral NP distribution in lung tissues section from infected hamsters treated with DMSO or propranolol, at 4 dpi was observed by Immunofluorescence staining of fixed lung tissues for SARS-COV-2 NP and cell nuclei, respectively. Representative images of viral NP distribution in lung tissues section from infected hamsters treated with DMSO or propranolol, at 4 dpi were analyzed, with SARS-COV-2 NP and cell nuclei stained.

### Results

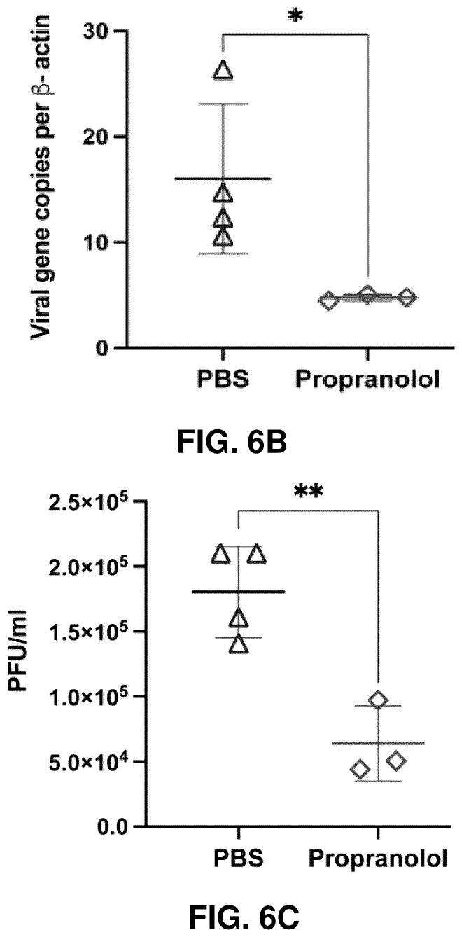

A golden Syrian hamster model to evaluate the in vivo efficacy of propranolol on SARS-COV-2 infection. Because propranolol was approved for medical use over than 50 years and already developed the injection dose form, the drug was delivered through intraperitoneal injection. In wild-type infected hamster, the first dose of 40 mg/kg of propranolol was given at 6 hpi, followed by another 3 doses delivered from 1 dpi to 3 dpi. At 4 dpi, animal lungs were harvested for evaluation of viral load (FIG. 6A).

Notably, propranolol decreased WT SARS-COV-2 genome copies (p<0.05) and infectious virus titer (p<0.01) in comparison with the mock-treated samples in lung tissues (FIGS. 6B-6C). The positive role of propranolol on WT SARS-COV-2 replication was further confirmed with immunofluorescence staining, which showed propranolol significantly reduced viral nucleocapsid protein expression in both bronchiolar and alveolar epithelia, indicating restricted virus spread.

We claim:

1. A method of reducing coronavirus replication in a cell, the method comprising contacting a cell comprising a replicative coronavirus genome with an effective amount of an anti-coronavirus agent that inhibits one or more gene expression products associated with the phosphatidate phosphatase (PAP) pathway in the cell to reduce the replication of the coronavirus in the cell relative to an untreated control cell.

2. The method of claim 1, wherein the anti-coronavirus agent that inhibits one or more gene expression products associated with the phosphatidate phosphatase (PAP) pathway comprises propranolol, or a derivative or variant of propranolol.

3. The method of claim 2, wherein the propranolol consists of L-isoform propranolol, or D-isoform propranolol, or comprises L-isoform propranolol and D-form propranolol.

4. The method of claim 3, wherein the propranolol is at a concentration of between about 10 μM and 500 μM, inclusive, optionally wherein the propranolol is at a concentration of about 100 μM.

5. The method of claim 1, wherein the cell is a human cell.

6. The method of claim 1, wherein the contacting is in vivo.

7. The method of claim 1, wherein the coronavirus is selected from the group consisting of Severe Acute Respiratory Syndrome (SARS)-CoV-2 wild-type, SARS-COV-2 Alpha variant B.1.1.7, SARS-COV-2 Beta variant B.1.351, SARS-COV-2 Gamma variant P.1, SARS-COV-2 B.1.617, SARS-COV-2 Kappa variant B.1.617.1, SARS-COV-2 Mu variant B.1.621, SARS-COV-2 Delta variant B.1.617.2, SARS-COV-2 B.1.617.3, SARS-COV-2 Omicron variant B.1.1.529, SARS-COV-2 Omicron variant BA.1+R346K, SARS-COV-2 Omicron variant BA.2, SARS-COV-2 Omicron variant BA.3, SARS-COV-2 Omicron variant BA.4, SARS-COV-2 Omicron variant BA.5, and Middle East respiratory syndrome (MERS-CoV).

8. A method of treating or preventing one or more symptoms of a coronavirus infection in a subject, the method comprising
administering to a subject in need thereof an effective amount of an anti-coronavirus agent that inhibits one or more gene expression products associated with the phosphatidate phosphatase (PAP) pathway in the subject to reduce the replication of the coronavirus in the subject relative to an untreated control subject.

9. The method of claim 8, wherein the anti-coronavirus agent that inhibits one or more gene expression products associated with the phosphatidate phosphatase (PAP) pathway comprises propranolol, or a derivative or variant of propranolol.

10. The method of claim 9, wherein the propranolol consists of L-isoform propranolol, or D-isoform propranolol, or comprises L-isoform propranolol and D-form propranolol.

11. The method of claim 8, wherein the propranolol is administered to the subject in an amount between about 0.01 mg/kg body weight of the subject and about 100 mg/kg body weight of the subject, inclusive,
optionally wherein the propranolol is in an amount between about 1 mg/kg body weight of the subject and 40 mg/kg body weight of the subject, inclusive.

12. The method of claim 8, wherein the subject does not have a disease or disorder selected from the group consisting of hypertension, angina pectoris, myocardial infarction, tachycardia, anxiety, panic attacks, hyperthyroidism, portal hypertension, esophageal variceal bleeding and ascites, post-traumatic stress disorder (PTSD), aggressive behavior associated with brain injuries, essential tremor, migraine and/or cluster headache, hyperhidrosis, infantile hemangioma, glaucoma, thyrotoxicosis, hypertrophic cardiomyopathy and atrial fibrillation.

13. The method of claim 8, wherein the coronavirus is selected from the group consisting of Severe Acute Respiratory Syndrome (SARS)-CoV-2 wild-type, SARS-COV-2 Alpha variant B.1.1.7, SARS-COV-2 Beta variant B.1.351, SARS-COV-2 Gamma variant P.1, SARS-COV-2 B.1.617, SARS-COV-2 Kappa variant B.1.617.1, SARS-COV-2 Mu variant B.1.621, SARS-COV-2 Delta variant B.1.617.2, SARS-COV-2 B.1.617.3, SARS-COV-2 Omicron variant B.1.1.529, SARS-COV-2 Omicron variant BA.1+R346K, SARS-COV-2 Omicron variant BA.2, SARS-COV-2 Omicron variant BA.3, SARS-COV-2 Omicron variant BA.4, SARS-COV-2 Omicron variant BA.5, and Middle East respiratory syndrome (MERS-CoV).

14. The method of claim 8, wherein the one or more symptoms is selected from the group consisting of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

15. The method of claim 8, wherein the composition is administered via the oral, rectal or intravenous route.

16. The method of claim 8, wherein the composition is administered in a form selected from the group consisting of powder, liquids, and suspensions.

17. The method of claim 8, wherein the composition is administered in combination with another therapeutic, prophylactic, or diagnostic agent,
optionally wherein the one or more agent is selected from the group consisting of bronchodilators, corticosteroids, methylxanthines, phosphodiesterase-4 inhibitors, anti-angiogenesis agents, antimicrobial agents, antioxidants, anti-inflammatory agents, immunosuppressant agents, and anti-allergic agents.

18. The method of claim 8, wherein the composition is administered in combination with one or more agents selected from the group consisting of Nirmatrelvir-Ritonavir (PAXLOVID), Remdesivir (VEKLURY), Molnupiravir (LAGEVIRIO), Ribavirin with lopinavir-ritonavir and one or more corticosteroids, Interferon alfa-1, Convalescent-phase plasma, Ribavirin in combination with interferon alfa-2a and/or interferon alfa-2b, Mycophenolic acid, antiviral small molecule agent K22; dsRNA-activated caspase oligomerizer (DRACO) protein, SARS-COV PLpro inhibitor GRL0617, nucleoside analogue BCX4430, and 5-hydroxychromone derivatives, or combinations thereof.

19. The method of claim 8, wherein the composition is administered more than once, at an interval selected from the group consisting of once a day, twice a day, three times a day, once a week, twice a week, three times a week, once every two weeks, approximately once a month, once every two months and once every three months.

20. A method of treating or preventing one or more symptoms of COVID in a subject, the method comprising administering to a subject in need thereof an effective amount of propanolol, or a derivative or analog thereof, to prevent or reduce one or more symptoms of COVID in the subject relative to an untreated control subject,
wherein the subject does not have a disease or disorder selected from the group consisting of hypertension, angina pectoris, myocardial infarction, tachycardia, anxiety, panic attacks, hyperthyroidism, portal hypertension, esophageal variceal bleeding and ascites, post-traumatic stress disorder (PTSD), aggressive behavior associated with brain injuries, essential tremor, migraine and/or cluster headache, hyperhidrosis, infantile hemangioma, glaucoma, thyrotoxicosis, hypertrophic cardiomyopathy and atrial fibrillation, and
wherein the propranolol is administered to the subject in an amount between about 0.1 mg/kg body weight of the subject and about 40 mg/kg body weight of the subject, inclusive,
optionally wherein the propranolol is administered daily for a period of three days, or more than three days.
